(19) United States
(12) Patent Application Publication
(10) Pub. No.: US 2006/0063938 A1
(43) Pub. Date: Mar. 23, 2006

(54) Compounds to treat hiv infection and aids

(76) Inventors: Terrence R Burke, Bethesda, MD (US); Xuechun Zhang, Frederick, MD (US); Godwin C.G Pais, Frederick, MD (US); Evguenia Svarovskaia, Frederick, MD (US); Vinay K Pathak, Frederick, MD (US); Christophe Marchand, Bethesda, MD (US); Yves Pommier, Bethesda, MD (US)

Correspondence Address: MERCHANT & GOULD PC, P.O. BOX 2903, MINNEAPOLIS, MN, 55402-0903, US

(21) Appl. No.: 10/497,898

(22) PCT Filed: Dec. 6, 2002
(86) PCT No.: PCT/US02/39254

§ 371 Date: Mar. 28, 2005

Related U.S. Application Data
(60) Provisional application No. 60/339,137, filed on Dec. 7, 2001.

(51) Int. Cl. C07D 231/38; C07C 247/16

(52) U.S. Cl. 548/371.4; 562/405; 552/1; 548/530

(57) ABSTRACT

The present invention relates to compounds of formula I: useful HIV infection, AIDS, and other similar diseases. These compounds include inhibitors of the retroviral integrase enzyme that are useful in the treatment of HIV infection, AIDS, and other similar diseases characterized by integration of a retroviral genome into a host chromosome. The compounds of the invention are useful in pharmaceutical compositions and methods of treatment to reduce incorporation of a donor DNA into a receiving DNA.

This application is being filed as a PCT international patent application in the name of The Government of the United States of America, as represented by the Secretary, Department of Health and Human Services (applicant for all designations except the U.S.), and in the names of Terrence R. Burke, a U.S. citizen and resident; Xuechun Zhang, a Chinese citizen and U.S. resident; Godwin C. G. Pais, an Indian citizen and U.S. resident; Evguenia Svarovskaia, a Russian citizen and U.S. resident; Vinay K. Pathak, a U.S. citizen and resident; Christophe Marchand, a French citizen and U.S. resident; and Yves Pommier, a U.S. citizen and resident (applicants for the U.S. designation only), on 6 Dec. 2002, designating all countries.

### FIELD OF THE INVENTION

The present invention is directed to compounds useful in treatment of HIV infection and AIDS and more specifically to compounds that are capable of inhibiting retroviral integrase, an enzyme that integrates the HIV genome into the host's chromosomes with resulting persistent infection.

### BACKGROUND OF THE INVENTION

The retrovirus human immunodeficiency virus (HIV) is the etiological agent of acquired immune deficiency syndrome (AIDS). A common feature of retrovirus replication is the insertion by virally-encoded integrase of proviral DNA into the host cell genome, a required step in HIV replication in human cells. Integration is believed to be mediated by integrase in three steps. The first, assembly, produces a stable nucleoprotein complex with viral DNA sequences. The second, cleavage, removes two nucleotides from the 3′ termini of the linear proviral DNA. The third, strand transfer, covalently joins the recessed 3′ OH termini of the proviral DNA at a staggered cut made at the host target site. Repair synthesis of the resultant gap can be accomplished by cellular enzymes.

The pol gene of HIV includes one open reading frame encoding reverse transcriptase, integrase and an HIV protease. All three enzymes have been shown to be essential for the replication of HIV. Several antiviral compounds that inhibit one of these enzymes, including azidothymidine (AZT), slow or stop HIV replication and effectively treat AIDS.

Compounds that are effective inhibitors of retroviral integrase, that inhibit integrase-mediated strand transfer, that are effective inhibitors of integration of the viral genome, and/or are effective to reduce HIV levels, are needed for the treatment and prevention of disease characterized by HIV infection or replication, such as AIDS.

### SUMMARY OF THE INVENTION

The present invention relates to compounds of the formulas below, compositions, and methods useful in the treatment of HIV infection and more specifically compounds that are capable of inhibiting retroviral integrase, an enzyme that integrates the HIV genome into the host's chromosomes with resulting persistent infection.

The present invention relates to azido diketo carboxylate compounds of formula I: 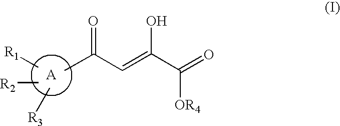
or a tautomer or pharmaceutically acceptable salt thereof. In formula I, at least one of R₁, R₂, or R₃ includes an azide moiety and A is an aromatic ring group.

Suitable aromatic ring groups, A, include aromatic carbocycles and heterocycles which can be fused to another aromatic ring group or other ring. Preferred aromatic ring groups include 5 and 6 carbon carbocyclic rings (e.g., phenyl ring) and 5 and 6 member heterocyclic rings, preferably including 1 or 2 nitrogen atoms (e.g. a pyrrolyl or pyrazolyl ring). Preferred aromatic ring groups include the 6 member aromatic carbocyclic phenyl ring.

Preferably, R₁, R₂, and R₃ can independently be —H, —N₃, halogen, —OH, —SH, —NH₂, —OR₅, —SR₅, —N(R₅)(R₅), or 1 to 6 carbon alkyl, alkene, or alkyne group, preferably containing an azide (—N₃) group. Such a 1 to 6 carbon alkyl, allcenyl, or alkynyl group it can include substituents such as one or more of hydroxyl, sulfhydryl, lower allcyl group (such as methyl or ethyl), lower alkoxy (such as methoxy or ethoxy), lower hydroxyalkyl (such as —CH₂OH or —CH₂CH₂OH), acyl, allyl, a halogenated alkyl group (e.g., a trifluoromethyl group), —C(O)—, —C(S)—, —C(O)H, ═O, —C(O)OH, halogen, and the like. One or more of R₁, R₂, or R₃ include an azide (—N₃) group. Preferably one or more of R₁, R₂, and R₃ is independently —N₃, or 1 to 6 carbon allcyl substituted with —N₃. Preferably, one or more of R₁, R₂, and R₃ is independently —N₃, —CH₂N₃, or —CH₂CH₂N₃.

Preferably, R₄ can be H or 1 to 6 carbon alkyl, alkene, or alkyne group, preferably containing an azide (—N₃) group. Such a 1 to 6 carbon alkyl, alkenyl, or alcynyl group it can include substituents such as one or more of hydroxyl, sulfhydryl, lower alkyl group (such as methyl or ethyl), lower alkoxy (such as methoxy or ethoxy), lower hydroxyalkyl (such as —CH₂OH or —CH₂CH₂OH), acyl, allyl, a halogenated alkyl group (e.g., a trifluoromethyl group), —C(O)—, —C(S)—, —C(O)H, ═O, —C(O)OH, halogen, and the like. Preferably R₄ is H or lower alkyl, such as methyl or ethyl.

Preferably, R₅ is a 1 to 6 carbon alkyl, alkenyl, or alkynyl group, which can include substituents such as one or more of —N₃, —OH, —SH, —NH₂, lower alkyl group (such as methyl or ethyl), lower alkoxy (such as methoxy or ethoxy), lower hydroxyalkyl (such as —CH₂OH or —CH₂CH₂OH), acyl, allyl, a halogenated alkyl group (e.g., a trifluoromethyl group), —C(O)—, —C(S)—, —C(O)H, ═O, —C(O)OH, halogen, and the like.

The present invention also includes a method of treating a patient who has, or in preventing a patient from getting, infection by HIV, AIDS, or AIDS related complex (ARC) and who is in need of such treatment which includes administration of a therapeutically effective amount of a compound of formula I: 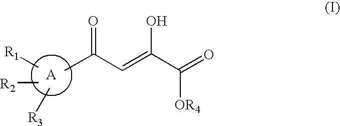
where A, R₁, R₂, R₃, and R₄ are as defined above, or a tautomer or pharmaceutically acceptable salt thereof.

In an embodiment, this method of treatment can be used where the disease is infection by HIV.

In an embodiment, this method of treatment can help prevent or delay the onset of infection by HIV.

In an embodiment, this method of treatment can be used where the disease is AIDS.

In an embodiment, this method of treatment can help prevent or delay the onset of AIDS.

In an embodiment, this method of treatment can be used where the disease is ARC.

In an embodiment, this method of treatment can help prevent or delay the onset of ARC.

In an embodiment, this method of treatment can treat an existing disease, such as those listed above.

In an embodiment, this method of treatment can prevent a disease, such as those listed above, from developing.

In an embodiment, this method of treatment can employ therapeutically effective amounts: for oral administration from about 0.1 mg/day to about 1,000 mg/day; for parenteral, sublingual, intranasal, intrathecal administration from about 0.5 to about 100 mg/day; for depo administration and implants from about 0.5 mg/day to about 50 mg/day; for topical administration from about 0.5 mg/day to about 200 mg/day; for rectal administration from about 0.5 mg to about 500 mg.

In an embodiment, this method of treatment can employ therapeutically effective amounts: for oral administration from about 1 mg/day to about 100 mg/day; and for parenteral administration from about 5 to about 50 mg daily.

In an embodiment, this method of treatment can employ therapeutically effective amounts for oral administration from about 5 mg/day to about 50 mg/day.

The present invention also includes a pharmaceutical composition which includes a compound of formula I: 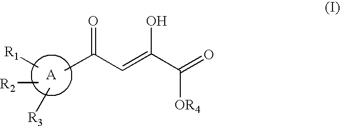
where A, R₁, R₂, R₃ and R₄ are as defined above, or a tautomer or pharmaceutically acceptable salt thereof; and an inert diluent or edible carrier.

The present invention also includes the use of a compound of formula I: 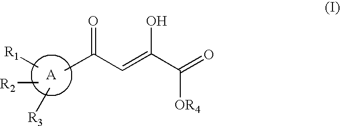
where A, R₁, R₂, R₃ and R₄ are as defined above, or a tautomer or pharmaceutically acceptable salt thereof, for the manufacture of a medicament for use in treating a patient who has, or in preventing a patient from getting, infection by HIV, AIDS, or ARC and who is in need of such treatment.

In an embodiment, this use of a compound of formula (I) can be employed where the disease is infection by HIV.

In an embodiment, this use of a compound of formula (I) can help prevent or delay the onset of infection by HIV.

In an embodiment, this use of a compound of formula (I) can be employed where the disease is AIDS.

In an embodiment, this use of a compound of formula (I) can help prevent or delay the onset of AIDS.

In an embodiment, this use of a compound of formula (I) can be employed where the disease is ARC.

In an embodiment, this use of a compound of formula (I) can help prevent or delay the onset of ARC.

In an embodiment, this use of a compound of formula (I) employs a pharmaceutically acceptable salt such as a base addition salt. Suitable base addition salts include those with metals or amines, such as alkali and alkaline metal earth metals or organic amines. Examples of metals used as cations include sodium, potassium, magnesium, calcium and the like. Suitable amines include N,N′-dibenzylethylenediamine, chloroprocaine, choline, diethanolamine, dicyclohexylamine, ethylenediamine, N-methylglucamine, and procaine.

The present invention also includes methods for inhibiting retroviral integrase activity, for inhibiting strand transfer catalyzed by retroviral integrase, for inhibiting incorporation of a donor strand DNA into a receiving strand DNA; for inhibiting HIV replication in a cell; for inhibiting HIV replication in an animal; and for treating or preventing a disease characterized by HIV infection or replication. These methods each include administration of a therapeutically effective amount of a compound of formula I: 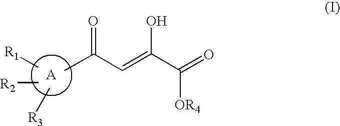
where A, R₁, R₂, R₃ and R₄ are as defined above, or a tautomer or pharmaceutically acceptable salt thereof.

The present invention also includes a method for inhibiting retroviral integrase activity, including exposing the retroviral integrase to an effective inhibitory amount of a compound of formula I: 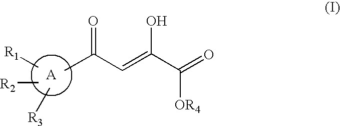
where A, R₁, R₂, R₃, and R₄ are as defined above, or a tautomer or pharmaceutically acceptable salt thereof.

In an embodiment, this method includes exposing the retroviral integrase to the compound in vitro.

In an embodiment, this method includes exposing the retroviral integrase to the compound in a cell.

In an embodiment, this method includes exposing the retroviral integrase to the compound in a cell in an animal.

In an embodiment, this method includes exposing the retroviral integrase to the compound in a human.

The present invention also includes a method for inhibiting strand transfer between a donor DNA strand and a receiving DNA strand, including exposing the reaction mixture to an effective inhibitory amount of a compound of formula I: 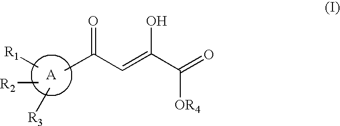
where A, R₁, R₂, R₃, and R₄ are as defined above, or a tautomer or pharmaceutically acceptable salt thereof

In an embodiment, this method employs an integration site recombinant or synthetic DNA as donor and or receiving DNA or employs cellular DNA as receiving DNA.

In an embodiment, this method exposes the reaction mixture in vitro.

In an embodiment, this method exposes the reaction mixture in a cell.

In an embodiment, this method exposes the reaction mixture in an animal cell.

In an embodiment, this method exposes the reaction mixture in a human cell.

The present invention also includes a method for inhibiting HIV replication in a cell, including administering to the cell an effective inhibitory amount of a compound of formula I: 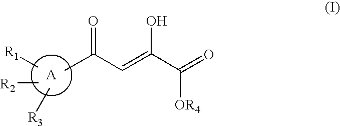
where A, R₁, R₂, R₃, and R₄ are as defined above, or a tautomer or pharmaceutically acceptable salt thereof.

In an embodiment, this method includes administering to an animal.

In an embodiment, this method includes administering to a human.

The present invention also includes a method for inhibiting the replication of HIV or reducing HIV burden in an animal, including administering to the animal an effective inhibitory amount of a compound of formula I: 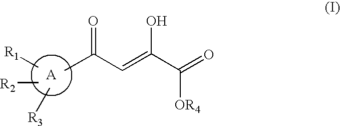
where A, R₁, R₂, R₃, and R₄ are as defined above, or a tautomer or pharmaceutically acceptable salt thereof.

In an embodiment, this method includes administering to a human.

The present invention also includes a method for treating or preventing a disease characterized by HIV integration or replication including administering to a patient an effective therapeutic amount of a compound of formula I: 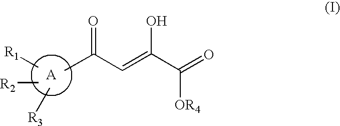
where A, R₁, R₂, R₃, and R₄ are as defined above, or a tautomer or pharmaceutically acceptable salt thereof.

In an embodiment, this method employs a compound at a therapeutic amount in the range of from about 0.1 to about 1000 mg/day.

In an embodiment, this method employs a compound at a therapeutic amount in the range of from about 15 to about 1500 mg/day.

In an embodiment, this method employs a compound at a therapeutic amount in the range of from about 1 to about 100 mg/day.

In an embodiment, this method employs a compound at a therapeutic amount in the range of from about 5 to about 50 mg/day.

In an embodiment, this method can be used where the disease is AIDS.

In an embodiment, this method can be used where the disease is HIV infection.

The present invention also includes a composition including retroviral integrase complexed with a compound of formula I: 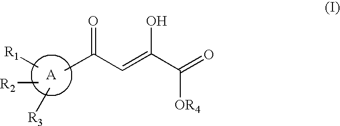
where A, R₁, R₂, R₃, and R₄ are as defined above, or a tautomer or pharmaceutically acceptable salt thereof

The present invention also includes a method for producing a retroviral integrase complex including exposing retroviral integrase to a compound of formula I: 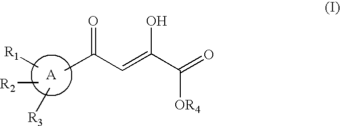
where A, R₁, R₂, R₃, and R₄ are as defined above, or a tautomer or pharmaceutically acceptable salt thereof, in a reaction mixture under conditions suitable for the production of the complex.

In an embodiment, this method employs exposing in vitro.

In an embodiment, this method employs a reaction mixture that is a cell. The present invention also includes a component kit including component parts capable of being assembled, in which at least one component part includes a compound of formula (I) enclosed in a container.

In an embodiment, this component kit includes lyophilized compound, and at least one further component part includes a diluent.

The present invention also includes a container kit including a plurality of containers, each container including one or more unit dose of a compound of formula I: 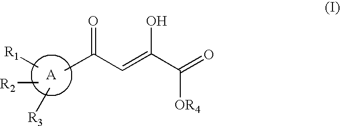
where A, R₁, R₂, R₃, and R₄ are as defined above, or a tautomer or pharmaceutically acceptable salt thereof.

In an embodiment, this container kit includes each container adapted for oral delivery and includes a tablet, gel, or capsule.

In an embodiment, this container kit includes each container adapted for parenteral delivery and includes a depot product, syringe, ampoule, or vial.

In an embodiment, this container kit includes each container adapted for topical delivery and includes a patch, medipad, ointment, or cream.

The present invention also includes an agent ldt including a compound of formula I: 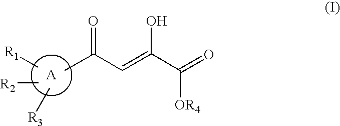
where A, R₁, R₂, R₃, and R₄ are as defined above, or a tautomer or pharmaceutically acceptable salt thereof; and one or more therapeutic agents such as nucleoside analog reverse transcriptase inhibitors, non-nucleoside reverse transcriptase inhibitors, protease inhibitors, other antivirals, immunomodulators, anti-infectives.

The present invention also includes a composition including: a compound of formula I: 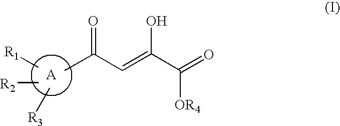
where A, R₁, R₂, R₃, and R₄ are as defined above, or a tautomer or pharmaceutically acceptable salt thereof; and an inert diluent or edible carrier.

In an embodiment, this composition includes a carrier that is an oil.

The present invention also includes a composition including: a compound of formula I: 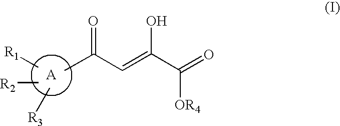
where A, R₁, R₂, R₃, and R₄ re as defined above, or a tautomer or pharmaceutically acceptable salt thereof; and a binder, excipient, disintegrating agent, lubricant, or gildant.

The present invention also includes a composition including: a compound of formula I: 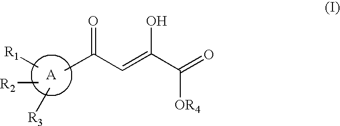
where A, R₁, R₂, R₃, and R₄ are as defined above, or a tautomer or pharmaceutically acceptable salt thereof; disposed in a cream, ointment, or patch.

The present invention provides compounds, compositions, kits, and methods for inhibiting retroviral integrase-mediated strand transfer, incorporation of a donor DNA into a receiving DNA, or other integrase activity. More particularly, the compounds, compositions, and methods of the invention are effective to inhibit the integration of a retroviral genome into a host chromosome and to treat or prevent any human or veterinary disease or condition associated with a retroviral integration.

The compounds, compositions, and methods of the invention are useful for treating humans who have infection by HIV, AIDS, or ARC.

The compounds of the invention possess retroviral integrase inhibitory activity. The inhibitory activities of the compounds of the invention are readily demonstrated, for example, using one or more of the assays described herein or known in the art.

### BRIEF DESCRIPTION OF THE FIGURES

FIGS. 1A through 1C represent (A) the oligonucleotides employed in, (B) reaction scheme for, and (C) results of an in vitro integration assay.

FIG. 2 illustrates inventive and control diketo carboxylates.

FIG. 3. Single cycle assay for determination of Anti-HIV-1 activity of integrase inhibitors. Structure of an HIV-1 based vector, pNluc, is shown. The vector contains two LTRs (long terminal repeats), functional gag (group-antigen) and pol (polymerase) genes and an inactivated env (*env, envelope). The vector also contain the firefly luciferase gene that was inserted into the HIV-1 nef gene. VSV-G, vesicular stomatitis virus G protein encoding gene that is expressed from a CMV promoter. The 293T cells were used to produce infectious pNluc virus. The 293T cells were also used as targets for infection in the absence or presence of tested inhibitor compounds. The infected cells were lysed and the luciferase activities present in the lysates were determined.

FIG. 4. Determination of IC50 values for P10 and azido group containing compounds. The IC50 determinations were performed as described in the text. All luciferase activities were normalized to the cells that were infected in the absence of any inhibitor in parallel experiments. Each experiment was repeated at least three times; the error bars represent the standard error of the mean.

FIG. 5. Determination of inhibition of cell proliferation by P10 and azido group containing compounds. The cell proliferation inhibition assays were performed as described in the text. The absorbance values in the presence of the XTT substrate were normalized to the cells that were maintained in the absence of any inhibitor in parallel experiments. Each experiment was repeated at least three times; the error bars represent the standard error of the mean.

FIG. 6. Determination of inhibition of replication competent HIV-1 by B compound. HIV-1 infection was carried out in the presence of 25 μM B compound, 100 nM AZT, or in the absence of any drugs. Uninfected H9 culture was used as a negative control. Inhibition of replication competent HIV-1 was evaluated by p24 antigen ELISA every 3 days for 15 days. Each time point of each culture was evaluated in duplicate.

### DETAILED DESCRIPTION OF THE INVENTION

The present invention relates to compounds of formula I: 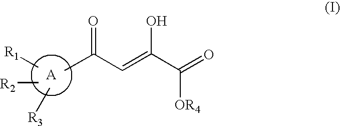
where A, R₁, R₂, R₃, and R₄ are as defined above, or a tautomer or pharmaceutically acceptable salt thereof.

When any variable (e.g., R₃, R₄, etc.) occurs more than one time in any constituent or in formula I, its definition on each occurrence is independent of its definition at every other occurrence. Also, combinations of substituents and/or variables are permissible only if such combinations result in stable compounds.

Particular compounds of structural formula I include: 4-(3,5-bis-azidomethyl-phenyl)-2-hydroxy-4-oxo-but-2-enoic; 4-(3-Azidomethyl-phenyl)-2-hydroxy-4-oxo-but-2-enoic acid; 4-(3-Azido-phenyl)-2-hydroxy-4-oxo-but-2-enoic acid; 4-(3,5-bis-azidomethyl-phenyl)-2-hydroxy-4-oxo-but-2-enoic acid; 4-(3-azidomethyl-phenyl)-2-hydroxy-4-oxo-but-2-enoic acid; 4-(3,5-Bis-azidomethyl-phenyl)-2-hydroxy-4-oxo-but-2-enoic acid; 4-(3-Azido-phenyl)-2-hydroxy-4-oxo-but-2-enoic acid; or a tautomer or a pharmaceutically acceptable salt thereof.

One embodiment of the present invention is a compound of structural formula: 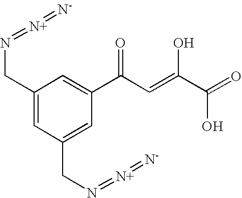
or a tautomer or a pharmaceutically acceptable salt thereof.

One embodiment of the present invention is a compound of structural formula: 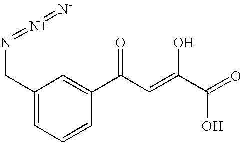
or a tautomer or a pharmaceutically acceptable salt thereof.

One embodiment of the present invention is a compound of structural formula: 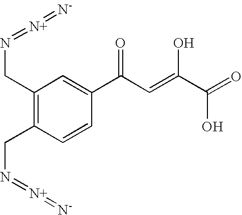
or a tautomer or a pharmaceutically acceptable salt thereof.

One embodiment of the present invention is a compound of structural formula: 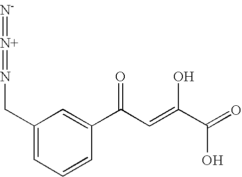
or a tautomer or a pharmaceutically acceptable salt thereof.

Preferably A is pyrrolyl, pyrazolyl, or phenyl. Preferably A is phenyl. Preferably, R₁, R₂, and R₃ are —H, —N₃, or 1 to 6 carbon alkyl substituted with —N₃, such as —CH₂N₃, or —CH₂CH₂N₃. One or more of R₁, R₂, or R₃ include an azide (—N₃) group. Preferably R₄ is H, —CH₃, or —CH₂C₃, preferably —H.

The compounds of formula (I) can be prepared by one skilled in the art without more simply by knowing the chemical structure of the compound. The chemistry is known to those skilled in the art. In fact, there is more than one process to prepare the compounds of the invention. Specific methods for preparing certain embodiments of the compounds of the invention are described in the Examples hereinbelow.

One example of a method of preparing compounds of the invention is given by the general procedure listed in Scheme 1:

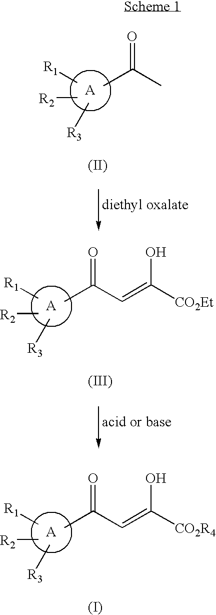

An azide containing substituent, R₁, R₂ or R₃, can be formed by brominating a suitable alkyl compound to form the corresponding alkyl bromide. The alkyl bromide can be treated with sodium azide to form the azide ketone (II), which is then converted to the ester form of the diketo carboxylate (III). Aromatic ketone (II) reacts with an oxalate to form an ester form of the diketo carboxylate (III). This ester is treated with base or acid to form the compound of formula I.

The compounds of the invention may contain geometric or optical isomers as well as tautomers. Thus, the invention includes all tautomers and pure geometric isomers, such as the E and Z geometric isomers, as well as mixtures thereof. Furthermore, the invention includes pure enantiomers and diastereomers as well as mixtures thereof, including racemic mixtures. The individual geometric isomers, enantiomers, or diastereomers may be prepared or isolated by methods known in the art.

As is recognized by workers in this field, the diketo-acid/ester compounds of the present invention exist as tautomers, and thus by using the phrase “and tautomers thereof” in describing compounds of structural formula (I), Applicants also intend the following tautomeric forms of the same compound (Ia) and (Ib):

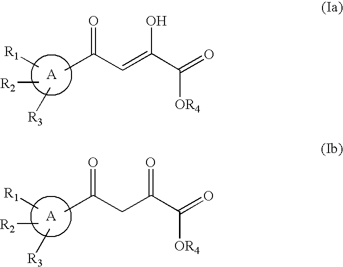

By naming or referring to compound (I) and tautomers thereof, it is understood for the purposes of the present application that the tautomers (Ia) and (Ib) are also intended. Similarly, be referring to compound (Ia), it is understood for the purposes of the present application that the tautomers (I) and (Ib) are also intended. The same holds true for references to tautomer (Ib).

When the compounds of formula (I) are carboxylic acids, they may form salts when reacted with bases. Pharmaceutically acceptable salts are preferred over the corresponding carboxylic acids of formula (I) since they produce compounds, which are more water soluble, stable and/or more crystalline. Pharmaceutically acceptable salts are any salt which retains the activity of the parent compound and does not impart any deleterious or undesirable effect on the subject to whom it is administered and in the context in which it is administered. Pharmaceutically acceptable salts include addition salts of both organic and inorganic bases. For examples of some acceptable salts, see Int. J Pharm., 33, 201-217 (1986) and J. Pharm. Sci., 66(1), 1, (1977).

Depending on the particular functionality of the compound of the present invention, pharmaceutically acceptable salts of the compounds of this invention include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, dicyclohexylamine, N-methylglucarnine, piperazine, tris(hydroxymethyl)aminomethane, and tetramethylammonium hydroxide. These salts may be prepared by standard procedures, e.g. by reacting a free acid with a suitable organic or inorganic base. Where a basic group is present, such as amino, an acidic salt, i.e. hydrochloride, hydrobromide, acetate, pamoate, and the like, can be used as the dosage form.

Also, in the case of an acid (——COOH) or alcohol group being present, pharmaceutically acceptable esters can be employed, e.g. acetate, maleate, pivaloyloxymethyl, and the like, and those esters known in the art for modifying solubility or hydrolysis characteristics for use as sustained release or prodrug formulations.

The present invention also provides for the use of a compound of structural formula (I) to make a pharmaceutical composition useful for inhibiting HIV integrase and in the treatment of AIDS or ARC.

Methods of the Invention

The compounds of the invention, and pharmaceutically acceptable salts thereof, are useful for treating humans or animals suffering from a condition characterized by a replication or integration of a retrovirus and for helping to prevent or delay the onset of such a condition. For example, the compounds are useful for treating infection by HIV, AIDS, or ARC. When treating or preventing these diseases, the compounds of the invention can either be used individually or in combination, as is best for the patient.

The compounds of the present inventions are useful in the inhibition of HIV integrase, the prevention or treatment of infection by human immunodeficiency virus (HIV) and the treatment of consequent pathological conditions such as AIDS. Treating AIDS or preventing or treating infection by HIV is defined as including, but not limited to, treating a wide range of states of HIV infection: AIDS, ARC (AIDS related complex), both symptomatic and asymptomatic, and actual or potential exposure to HIV. For example, the compounds of this invention are useful in treating infection by HIV after suspected past exposure to HIV by e.g., blood transfusion, exchange of body fluids, bites, accidental needle stick, or exposure to patient blood during surgery.

As used herein, the term “treating” means that the compounds of the invention can be used in humans with at least a tentative diagnosis of disease. The compounds of the invention will delay or slow the progression of the disease thereby giving the individual a more useful life span.

The term “preventing” means that the compounds of the present invention are useful when administered to a patient who has not been diagnosed as possibly having the disease at the time of administration, but who would normally be expected to develop the disease or be at increased risk for the disease. The compounds of the invention will slow the development of disease symptoms, delay the onset of the disease, or prevent the individual from developing the disease at all.

In treating or preventing the above diseases, the compounds of the invention are administered in a therapeutically effective amount. The therapeutically effective amount will vary depending on the particular compound used and the route of administration, as is well known.

In treating a patient displaying any of the diagnosed above conditions a physician may administer a compound of the invention immediately and continue administration indefinitely, as needed. Upon HIV infection or exposure, even though the patient does not have symptoms of disease, administration of the compounds of the invention may be started before symptoms appear, and treatment may be continued indefinitely to prevent or delay the onset of disease.

The compounds of this invention are useful in the preparation and execution of screening assays for antiviral compounds. For example, the compounds of this invention are useful for isolating enzyme mutants, which are excellent screening tools for more powerful antiviral compounds. Furthermore, the compounds of this invention are useful in establishing or determining the binding site of other antivirals to HIV integrase, e.g., by competitive inhibition. Thus the compounds of this invention are commercial products to be sold for these purposes.

The present invention further provides compounds, compositions, kits, and methods for inhibiting retroviral integrase enzyme activity and retroviral replication. Inhibition of retroviral integrase enzyme activity halts or reduces the integration of donor DNA into receiving DNA and reduces or halts retroviral replication.

Dosage Forms and Amounts

In accordance with the present invention there is further provided a method of treating and a pharmaceutical composition for treating HIV infection and AIDS. The treatment involves administering to a patient in need of such treatment a pharmaceutical composition comprising a pharmaceutical carrier and a therapeutically-effective amount of a compound of the present invention.

For these purposes, the compounds of the present invention may be administered orally, parenterally (including subcutaneous injections (SQ and depo SQ), intravenous (IV), intramuscular (IM and depo-IM), intrastemal injection or infusion techniques), sublingually, intranasally (inhalation), intrathecally, topically, or rectally, in dosage unit formulations containing conventional non-toxic pharmaceutically-acceptable carriers, adjuvants and vehicles. Dosage forms known to those of skill in the art are suitable for delivery of the compounds of the invention.

The terms “administration of” and or “administering a” compound should be understood to mean providing a compound of the invention, a prodrug of a compound of the invention, or a composition of the invention to the individual in need of treatment.

As used herein, the term “composition” is intended to encompass a product comprising the specified ingredients in the specified amounts, as well as any product which results, directly or indirectly, from combination of the specified ingredients in the specified amounts.

Compositions are provided that contain therapeutically effective amounts of the compounds of the invention. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

About 1 to 500 mg of a compound or mixture of compounds of the invention or a physiologically acceptable salt or ester is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 2 to about 100 mg, more preferably about 10 to about 30 mg of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

To prepare compositions, one or more compounds of the invention are mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

By “pharmaceutically acceptable” it is meant the carrier, diluent or excipient must be compatible with the other ingredients of the formulation and not deleterious to the recipient thereof.

Pharmaceutical carriers or vehicles suitable for administration of the compounds provided herein include any such carriers known to be suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions.

The compounds of the invention may be prepared with carriers that protect them against rapid elimination from the body, such as time-release formulations or coatings. Such carriers include controlled release formulations, such as, but not limited to, microencapsulated delivery systems. The active compound is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder.

The compounds and compositions of the invention can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compound of the invention. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The concentration of the compound is effective for delivery of an amount upon administration that lessens or ameliorates at least one symptom of the disorder for which the compound is administered. Typically, the compositions are formulated for single dosage administration. The concentration of active compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

The invention here is the new compounds of the invention and new methods of using the compounds of the invention. Given a particular compound of the invention and a desired dosage form, it is known how to prepare and administer the appropriate dosage form. However, the following routes of administration of compounds and compositions of the invention are offered as exemplary.

Pharmaceutical compositions of the invention may be in a form acceptable for oral (suspensions or tablets for example), parenteral (including subcutaneous injections (SQ and depo SQ), intravenous (IV), intramuscular (IM and depo-IM), intrastemal injection or infusion techniques), sublingual, intranasal (nasal spray for example), intrathecal, topical (ointment), rectal (suppository), or through implant administration for example.

When administered orally as a suspension, these compositions are prepared according to techniques well-known in the art of pharmaceutical formulation and may contain microcrystalline cellulose for imparting bulk, alginic acid or sodium alginate as a suspending agent, methylcellulose as a viscosity enhancer, and sweeteners/flavoring agents known in the art. As immediate release tablets, these compositions may contain microcrystalline cellulose, dicalcium phosphate, starch, magnesium stearate and lactose and/or other excipients, binders, extenders, disintegrants, diluents and lubricants known in the art.

If oral administration is desired, the compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action.

When administered orally, compounds of the invention can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, it is preferred that they be of the sustained release type so that the compounds of the invention need to be administered only once or twice daily.

The oral dosage forms are administered to the patient 1, 2, 3, or 4 times daily. It is preferred that the compounds of the invention be administered either three or fewer times, more preferably once or twice daily. Hence, it is preferred that the compounds of the invention be administered in oral dosage form. It is preferred that whatever oral dosage form is used, that it be designed so as to protect the compounds of the invention from the acidic environment of the stomach. Enteric coated tablets are well known to those skilled in the art. In addition, capsules filled with small spheres each coated to protect from the acidic stomach, are also well known to those skilled in the art.

When administered orally, an administered amount therapeutically effective to inhibit retroviral integrase activity, to inhibit retroviral integrase mediated strand transfer, to inhibit retroviral mediated incorporation of a donor DNA into a receiving DNA, to inhibit HIV replication, to inhibit, prevent, or treat HIV infection, to treat or prevent AIDS is from about 0.1 mg/day to about 1,000 mg/day. It is preferred that the oral dosage is from about 1 mg/day to about 100 mg/day. It is more preferred that the oral dosage is from about 5 mg/day to about 50 mg/day. It is understood that while a patient may be started at one dose, that dose may be varied over time as the patient's condition changes.

The compounds of this invention can be administered orally to humans in a dosage range of 1 to 1000 mg/kg body weight in divided doses. One preferred dosage range is 0.1 to 200 mg/kg body weight orally in divided doses. Another preferred dosage range is 0.5 to 100 mg/lcg body weight orally in divided doses. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known for example, as described in U.S. Pat. No. 4,522,811. The compounds of the invention can be administered parenterally, for example, by IV, IM, depo-IM, SC, or depo-SC. When administered parenterally, a therapeutically effective amount of about 0.5 to about 100 mg/day, preferably from about 5 to about 50 mg daily should be delivered. When a depot formulation is used for injection once a month or once every two weeks, the dose should be about 0.5 mg/day to about 50 mg/day, or a monthly dose of from about 15 mg to about 1,500 mg.

The compounds of the invention can be administered sublingually. When given sublingually, the compounds of the invention should be given one to four times daily in the amounts described above for IM administration.

The compounds of the invention can be administered intranasally. When given by this route, the appropriate dosage forms are a nasal spray or dry powder, as is known. The dosage of the compounds of the invention for intranasal administration is the amount described above for IM administration.

When administered by nasal aerosol or inhalation, these compositions are prepared according to techniques known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The compounds of the invention can be administered intrathecally. When given by this route the appropriate dosage form can be a parenteral dosage form as is known. The dosage of the compounds of the invention for intrathecal administration is the amount described above for IM administration.

The compounds of the invention can be administered topically. When given by this route, the appropriate dosage form is a cream, ointment, or patch. Because of the amount of the compounds of the invention to be administered, the patch is preferred. When administered topically, the dosage is from about 0.5 mg/day to about 200 mg/day. Because the amount that can be delivered by a patch is limited, two or more patches may be used. The number and size of the patch is not important, what is important is that a therapeutically effective amount of the compounds of the invention be delivered as is known. The compounds of the invention can be administered rectally by suppository as is known. When administered by suppository, the therapeutically effective amount is from about 0.5 mg to about 500 mg.

When rectally administered in the form of suppositories, these compositions may be prepared by mixing the drug with a suitable non-irritating excipient, such as cocoa butter, synthetic glyceride esters of polyethylene glycols, which are solid at ordinary temperatures, but liquefy and/or dissolve in the rectal cavity to release the drug.

The compounds of the invention can be administered by implants as is known. When administering a compound of the invention by implant, the therapeutically effective amount is the amount described above for depot administration.

The compounds of the invention are used in the same manner, by the same routes of administration, using the same pharmaceutical dosage forms, and at the same dosing schedule as described above, for preventing disease or treating patients with HIV infection, AIDS, or ARC.

Additionally, the present invention is directed to a pharmaceutical composition comprising a therapeutically effective amount of a compound of the present invention in combination with a therapeutically effective amount of an AIDS treatment agent such as nucleoside analog reverse transcriptase inhibitors, non-nucleoside reverse transcriptase inhibitors, protease inhibitors, other antivirals, immunomodulators, anti-infectives, other antibiotics, or other medicines useful against HIV infection, AIDS, or ARC.

Suitable antivirals of all categories include Amprenivir, Abacavir, Acyclovir, Adefovir dipivoxil, Alpha Interferon, Retrovir, Ansamycin, beta-fluoro-ddA, Cidofovir, Curdlan sulfate, Cytovene, Ganciclovir, Delaviridine, Dideoxycytidine, Dideoxyinosine, Efavirenz, Famciclovir, Hypericin, Interferon Beta, Interferon alfa-n3, Indinavir, Lamivudine, Lobucavir, Nelfinavir, Nevirapine, Novapren, Phosphonoformate, Probucol, Ritonavir, Saquinavir, Didehydrodeoxythymidine, Valaciclovir, Virazole, Ribavirin, Zalcitabine, and Zidovudine (AZT).

Suitable immunomodulators include Bropirimine, Acemannan, interferons such as gamma interferon and alpha interferon, tumor necrosis factor, granulocyte macrophage colony stimulating factor, interleuldn-2, recombinant or soluble CD4.

Suitable anti-infectives include Clindamycin, Primaquine, Fluconazole, Pastille, Nystatin Pastille, Ornidyl, Eflornithine, Pentamidine, Isethionate, Trimethoprim, Trimethoprim/sulfa, Piritrexim, Pentamidine, Spiramycin, Trimetrexate.

The scope of combinations of the compounds of this invention with antivirals, immunomodulators, anti-infectives or vaccines is not limited to those listed above, but can include any combination with any pharmaceutical composition useful for the treatment of AIDS.

Preferred combinations are simultaneous or alternating treatments of with a compound of the present invention and an inhibitor of HIV protease and/or a non-nucleoside inhibitor of HIV reverse transcriptase. An optional fourth component in the combination is a nucleoside inhibitor of HIV reverse transcriptase, such as AZT, 3TC, ddC or ddI. Preferred HIV protease inhibitors include indinavir, nelfinavir, ritonavir, and saquinavir. Preferred non-nucleoside inhibitors of HIV reverse transcriptase include nevirapine and efavirenz.

In such combinations the compound of the present invention and other active agents may be administered separately or in conjunction. In addition, the administration of one element may be prior to, concurrent to, or subsequent to the administration of other agent(s).

It should be apparent to one skilled in the art that the exact dosage and frequency of administration will depend on the particular compounds of the invention administered, the particular condition being treated, the severity of the condition being treated, the age, weight, general physical condition of the particular patient, and other medication the individual may be taking as is well known to administering physicians who are skilled in therapy of retroviral infections, diseases, and associated disorders.

Inhibition of Retroviral Integrase

Various assays for measuring activities of retroviral integrase are known. For example, assays for the strand transfer activity of integrase can be conducted according to Wolfe, A.L. et al., J. Virol. 70, 1424 (1996), and Farnet, C. M. and Bushman F. D. (1997) Cell; 88, 483 for recombinant integrase and preintegration complexes. In addition, assays for the inhibition of acute HIV infection of T-lymphoid cells can be conducted according to Vacca, J. P. et al., (1994), Proc. Natl. Acad. Sci. USA 91, 4906.

Methods of determining inhibition of retroviral integrase in vitro and in vivo can also be determined, for example, in accordance with procedures given in the examples included herein. See Mazumder, A., Nearnati, N., Sunder, S., Owen, J., and Pommier, Y. Retroviral Integrase: A Novel Target in Antiviral Development; Basic In Vitro Assays with the Purified Enzyme. In: Antiviral Methods and Protocols, D. Kinchington and R. Schinazi (eds.), pp. 327-335. Totowa, N.J.: The Humana Press, Inc., 1999, and also Marchand, C., Nearnati, N., and Pornmier, Y. In vitro human immunodeficiency virus type 1 integrase assays. Methods Enzymol. 340: 624-633, 2001.

Definitions And Conventions

The definitions and explanations below are for terms as used throughout this entire document including both the specification and claims.

Definitions All temperatures are in degrees Celsius.

Chromatography (column and flash chromatography) refers to purification/separation of compounds expressed as (support, eluent). It is understood that the appropriate fractions are pooled and concentrated to give the desired compound(s).

HPLC refers to high pressure liquid chromatography.

Pharmaceutically acceptable refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability.

Saline refers to an aqueous saturated sodium chloride solution.

A therapeutically effective amount is defined as an amount effective to reduce or lessen at least one symptom of the disease being treated or to reduce or delay onset of one or more clinical markers or symptoms of the disease.

The present invention may be better understood with reference to the following examples. The following examples are also provided to further illustrate details for the preparation and use of the compounds of the present invention. These examples are intended to be representative of specific embodiments of the invention, and are not intended as limiting the scope of the invention. Furthermore, the compounds described in the following examples are not to be construed as forming the only genus that is considered as the invention, and any combination of the compounds or their moieties may itself form a genus. Those skilled in the art will readily understand that known variations of the conditions and processes of the following preparative procedures can be used to prepare these compounds.

### EXAMPLES

Example 1: Extracellular Evaluation of Compounds of the Invention Against Recombinant HIV Integrase

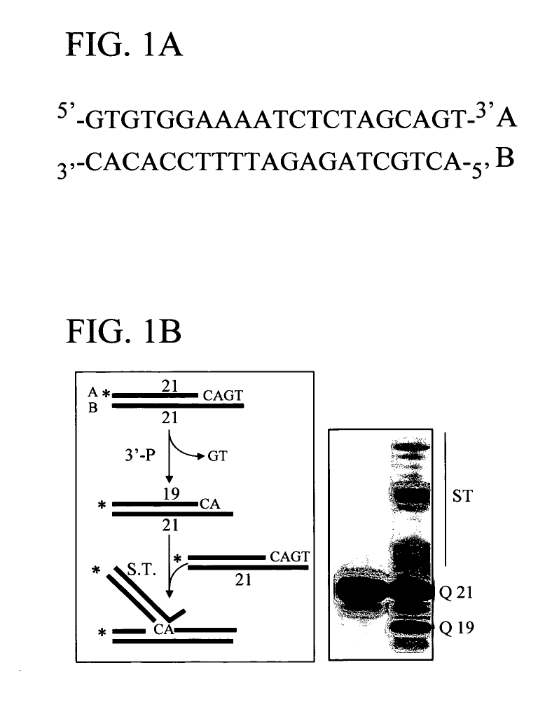

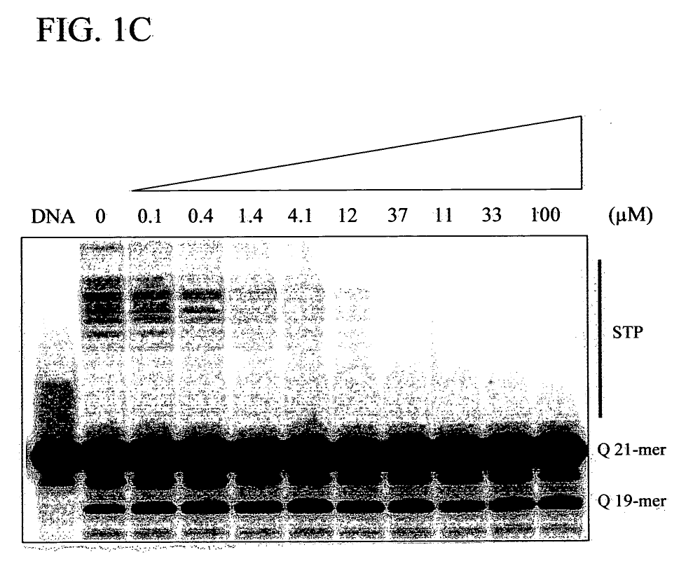

Compounds of the invention were tested for their ability to inhibit HIV-1 integrase in vitro. In the assay, a 21-mer double-stranded DNA oligonucleotide (oligonucleotide *A 5′-end-labeled annealed to oligonucleotide B; FIG. 1A), corresponding to the last 21 bases of the U5 viral LTR, is used to follow both the 3′-processing and 3′-end joining (strand transfer) steps of the integration reaction. Oligonucleotide A is 5′-end-labeled by T4-Polynucleotide Kinase (Gibco BRL/Life Technologies, Rockville, Md.). 10 pmoles of oligonucleotide A is incubated at 37° C. for 30 min in 50 μl of 1X kinase buffer containing 10 μCi of γ-ATP (Amersham Pharmacia Biotech, Piscataway, N.J.) and 10 units of kinase. The labeling solution is then applied to the top of a G25 Quick Spin column (Boehringer Mannheim, Indianapolis, Ind.) and the filtrate is annealed with 20 pmoles of oligonucleotide B (complementary strand) for 5 min at 95° C. and 30 min at 37° C.

In the 3′-processing reaction, integrase liberates a GT dinucleotide at the 3′-end of the labeled strand resulting in the generation of a 19-mer labeled product. The strand transfer (3′-end joining) reaction consists of the insertion of a 3′-processed oligonucleotide into another DNA target. This strand transfer step leads to higher and lower molecular weight species migrating slower and faster respectively, than the original 21-mer substrate (FIG. 1B). The higher molecular weight species (STP) are generally used to evaluate strand transfer (integration).

For inhibitor testing, a DNA-enzyme complex is preformed by mixing 400 nM HIV-1 integrase and 5 nM 5′-labeled double-stranded DNA template (*A/B) in a buffer containing 25 mM MOPS, pH 7.2, 7.5 mM MnCl₂, 14.3 mM β-mercaptoethanol, and 0.1 mg/ml Bovine Serum Albumin (BSA) for 15 min on ice. The integration reaction is then initiated by addition of the compound of the invention and continued in a total volume of 10 μl for 30 min at 37° C. The reaction is stopped by adding the same volume of electrophoresis denaturing dye containing 99% formamide (Sigma-Aldrich, Milwaukee, Wis.), 1% SDS, 0.2 mg/ml bromophenol blue (Sigma-Aldrich, Milwaukee, Wis.) and 0.2 mg/ml xylene cyanol blue (Sigma-Aldrich, Milwaukee, Wis.). Samples are then heated for 5 min at 95° C. and loaded on 20% 19/1 acrylamnide denaturing gel, Accugel (National Diagnostics, Atlanta, Ga.) containing 7 M urea (Gibco BRL/Life Technologies, Rockville, Md.) in 1 X TBE (Gibco BRL/Life Technologies, Rockville, Md.). Gels were exposed overnight and analyzed using a Molecular Dynamics Phosphorinmager (Sunnyvale, Calif.).

Diketo acids have been described previously to selectively inhibit the strand transfer step of the integration reaction. See Hazuda, D. J., Felock, P., Witmer, M., Wolfe, A., Stillmock, K., Grobler, J. A., Espesath, A., Gabryelski, L., Schlelf, W., Blau, C., and Miller, M. D. Inhibitors of strand transfer that prevent integration and inhibit HIV-1 replication in cells. Science 287: 646-650, 2000.

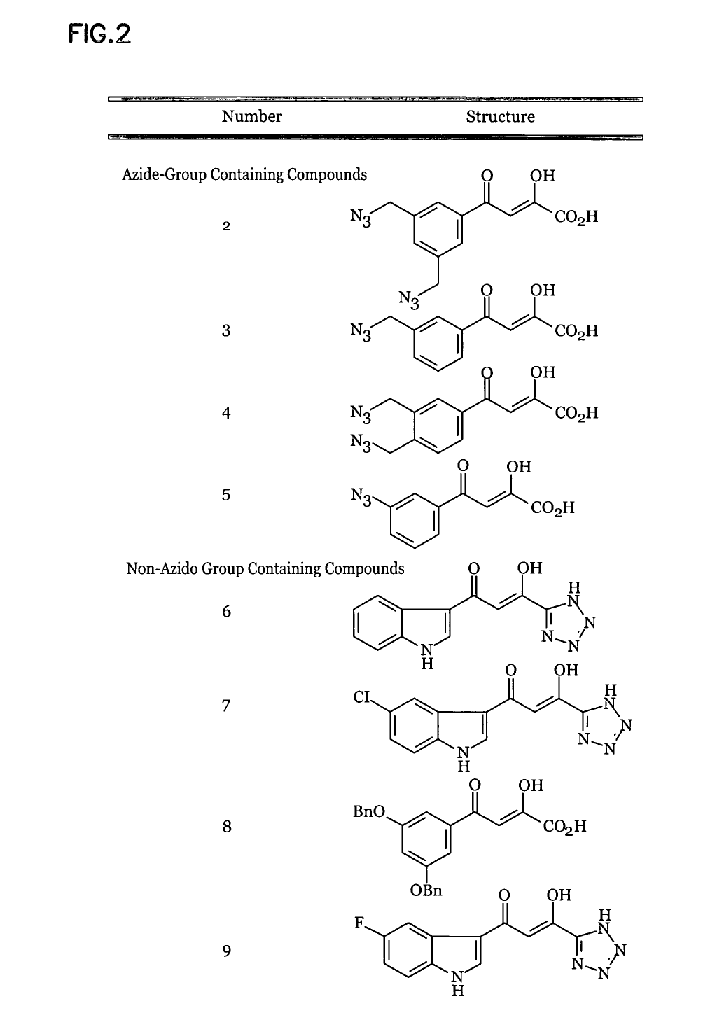

A typical experiment corresponding to the inhibition of HIV- 1 integrase by increasing concentrations of compound P10 (FIG. 1C), reveals an IC₅₀ in the micromolar range for the strand transfer reaction whereas, the 3′-processing step is unaffected by the compound even at concentrations up to the millimolar range. The results are summarized in Table 1 below. The structures of the inventive and control diketo carboxylates are illustrated in FIG. 2.

TABLE 1
In vitro inhibition of HIV-1 integrase by diketo acid derivatives

| | | |
|---|---|---|
| | IC50 3′- | IC50 Strand |
| Compound | Processing (μM) | transfer (μM) |

Azido Group-Containing Compounds

| | | |
|---|---|---|
| 2 | >100 | 2.0, 2.8 (n = 2) |
| 3 | >100 | 1.53 ± 0.27 (n = 3) |
| 4 | >100 | 0.26, 0.15 (n = 2) |
| 5 | >100 | 5.3, 5.2 (n = 2) |

Non-Azido Group-Containing Compounds

| | | |
|---|---|---|
| 6 | >100 | 1.9, 2.0 (n = 2) |
| 7 | 78 | 0.65 ± 0.19 (n = 4) |
| 8 | >1000 | 1.03 ± 0.25 (n = 3) |
| 9 | 79 | 1.8 |
| 10 | 7.2, 7.5 (n = 2) | 1.28 ± 0.38 (n = 3) |
| 11 | 8.1, 5.5 (n = 2) | 2.2, 1.6 (n = 2) |
| 12 | >100 | 6.9 |
| 13 | 48, 80 | 0.52 ± 0.10 (n = 3) |
| 14 | 82 | 6.56 ± 0.81 (n = 3) |
| 15 | >100 | 9.0, 11.0 (n = 2) |
| 16 | >100 | 7.8, 15.0 |
| 17 | 9 | 4.7 |
| 18 | 65, 100 | 0.35 ± 0.13 (n = 3) |
| 19 | >100 | 2.7, 2.1 (n = 2) |

Compounds of the invention, azido group-containing diketo acids, given as A-D in Table 1 are very potent against HIV-1 integrase in vitro with IC₅₀ values in the micromolar range and remain selective for the inhibition of the strand transfer step. The compound bearing two azido groups in the meta position (C) appears to be more potent than its equivalent in the meta position (A).

These results demonstrate that azido group-containing diketo acids can efficiently inhibit HIV-1 integrase in vitro, without any loss in selectivity for the strand transfer step of the integration reaction.

Example 2: Whole Cell Antiviral Evaluation of Compounds of the Invention

A series of compounds were tested for their ability to inhibit HIV-1 infection. Compounds were pre-selected based on their ability to inhibit the integration reaction in vitro by 50% at <10 μM concentrations.

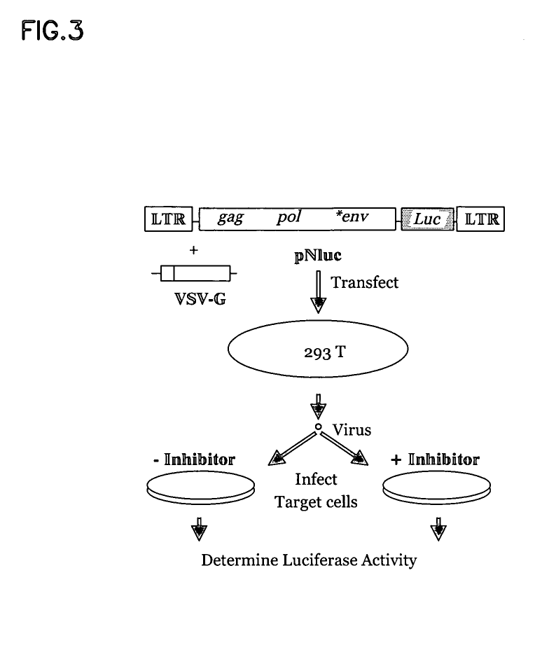

A single cycle replication assay was utilized to evaluate the antiviral activity of selected compounds (FIG. 3). An envelope deficient HIV-1 based retroviral vector containing the firefly luciferase reporter gene (pNluc) was cotransfected with vesicular stomatitis virus G glycoprotein (VSV-G) expressing plasmid (CMV-VSVG) into 293T cells. Virus harvested from transfected cells was used to infect target cells in the presence or absence of the compounds tested. The ability of the compounds to inhibit viral replication was measured by determining the amount of luciferase activity in the infected cells. Initially, compounds were tested at 25 μM concentrations. The results are summarized in Table 2.

TABLE 2
Inhibition of HIV-1 infection by
azido-group containing compounds.

| | | Luciferase Activity Relative to | 50% Inhibitory |
|---|---|---|---|
| Number | Compound | No Drug Control (Avg ± S.E.) | Conc. (IC50) |
| 1 | No Drug | 100 ± 22% | — |

Azido-Group Containing Compounds

| | | | |
|---|---|---|---|
| 2 | A | 5 ± 6% | 1.6 μM |
| 3 | B | 15 ± 7% | 0.5 μM |
| 4 | C | 36 ± 7% | 19 μM |
| 5 | D | 137 ± 12% | N.D.* |

Non-Azido Group Containing Compounds

| | | | |
|---|---|---|---|
| 6 | P4 | 116 ± 27% | N.D.* |
| 7 | P8 | 125 ± 11% | N.D.* |
| 8 | P10 | 7 ± 2% | 2.3 μM |
| 9 | P18 | 59 ± 13% | N.D. |
| 10 | P20 | 133 ± 35% | N.D.* |
| 11 | P22 | 98 ± 28% | N.D.* |
| 12 | P23 | 104 ± 25% | N.D.* |
| 13 | P24 | 90 ± 15% | N.D.* |
| 14 | P31 | 61 ± 17% | N.D. |
| 15 | E | 99 ± 14% | N.D.* |
| 16 | F | 261 ± 5% | N.D.* |
| 17 | G | 24 ± 8% | 2.2 μM |
| 18 | H | 14 ± 3% | 0.6 μM |
| 19 | I | 19 ± 2% | N.D. |

Compound P10 was used as a control, because it was shown previously to inhibit HIV-1 integration in vitro and in vivo Hazuda, D. J. et. al., (2000), Science 287:646-650. In this assay, the P10 compound inhibited HIV-1 infection to 7% of the untreated control, indicating that it was active in inhibiting HIV-1 replication. Compound P8 was previously shown to inhibit the strand transfer reaction catalyzed by HIV-1 integrase in vitro. This compound did not display any antiviral activity in the in vivo assay. Three compounds (A-C) significantly inhibited HIV-1 infection. A structurally related compound lacking the azido groups (E) did not show antiviral activity, suggesting that azido groups could play a significant role in antiviral activity.

In addition to the 4 azido-group containing compounds, 14 additional compounds were also tested for their ability to inhibit HIV-1 infection in a single cycle assay. Among the compounds tested, only the P10 compound previously shown to inhibit HIV-1 replication and 3 additional compounds exhibited anti-HIV-1 activity. Interestingly, one of these three compounds, H, is structurally related to P10 but exhibits a significantly lower IC50 (0.6 μM) than P10 (2.3 μM), suggesting that it may be more effective at inhibiting HIV-1 replication than P10.

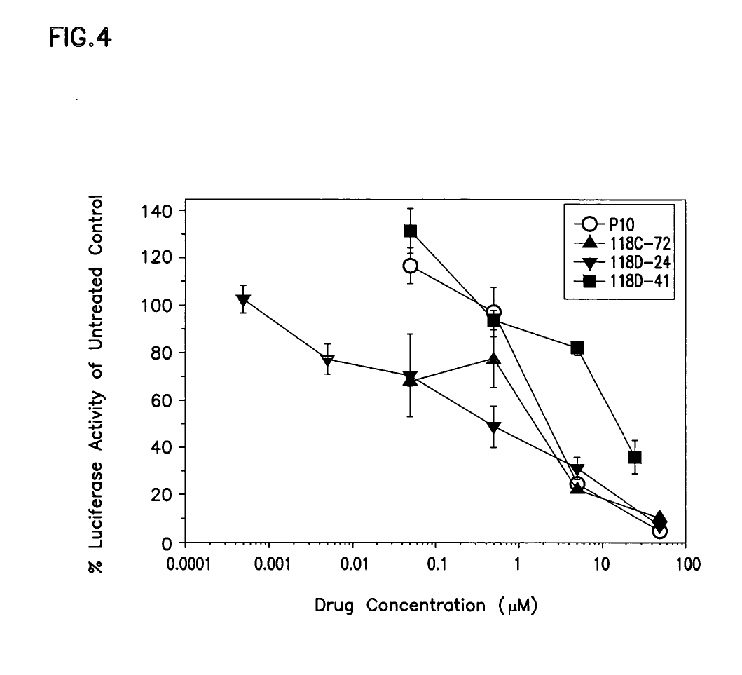

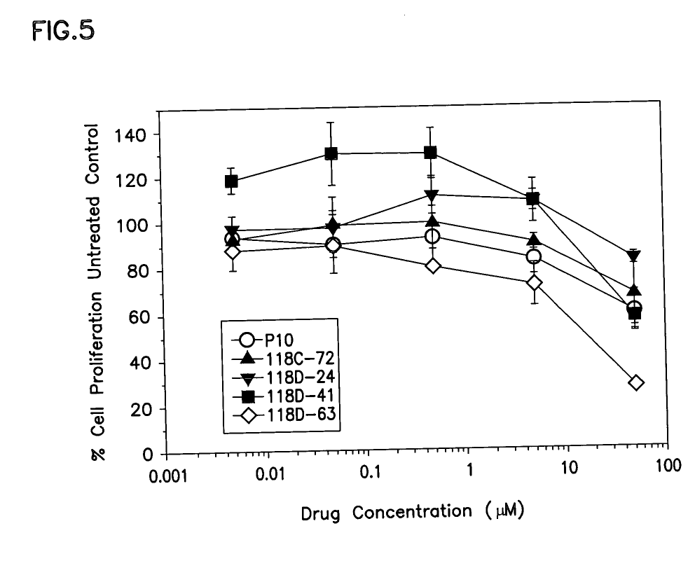

Additionally, concentrations of compounds that resulted in 50% inhibition of HIV-1 infection (IC50s) were determined for compounds that displayed antiviral activity at 25 μM concentrations (FIG. 4, Table 2). The single cycle HIV-1 replication assay was performed in the presence of a series of concentrations of the compounds (0.05 μM, 0.5 μM, 5 μM, and 50 μM). To determine a relative inhibition of HIV-1 infection at each concentration, luciferase activity of each sample was normalized to the untreated control. These experiments indicated that the IC50s for the azido group-containing compounds A and B were 1.6 μM and 0.5 μM, respectively. These IC50s were lower than the 2.3 μM IC50 for the P10 compound as determined in our experiments and the 2.5 μM IC50 reported previously Hazuda, D. J. et. al., (2000), Science 287:646-650.

To ensure that inhibition of luciferase activity in the presence of the compounds was indeed due to inhibition of HIV-1 infection and not due to the toxicity of these compounds to the target cells, we performed a cell toxicity assay in the presence of the same concentrations of compounds that were used for IC50 determination (FIG. 5). The azido-group containing compounds, A-C, did not display significant toxicity at 5 μM concentrations and had minimal toxicity, <50% inhibition, at 50 μM concentrations when the cells were maintained in the presence of the compounds for over 48 hours. One of the azido-group containing compounds, D, displayed 50% inhibition of cell proliferation at a concentration of approximately 19 μM.

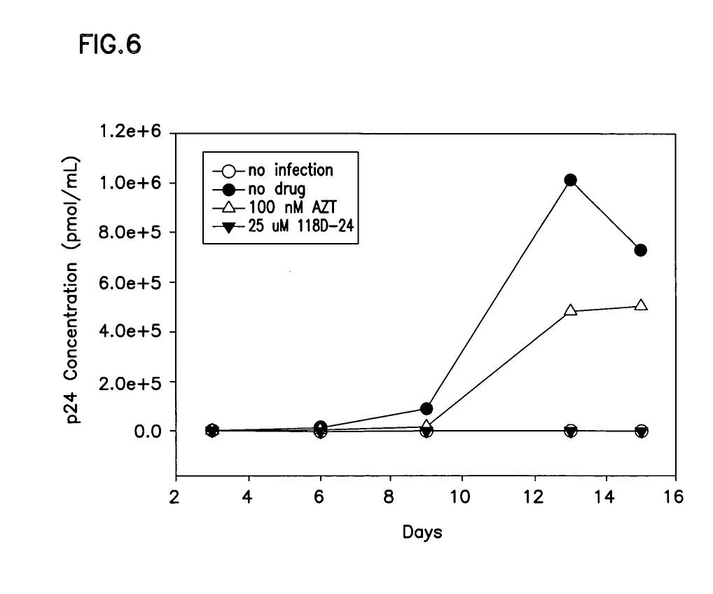

Inhibition of replication competent HIV-1 by B compound was evaluated at 25 μM concentration. 293T cells were transfected with pNL4-3, a plasmid that encodes full HIV-1 genome. Virus harvested from the transfected cells was used to infect H9 target cells. H9 infected cultures were carried out for 15 days in the absence or presence of 25 μM B compound and were assayed for p24 antigen every 3 days (FIG. 6). In addition, H9 infected cells were cultured in the presence of 100 nM AZT as a control. 100 nM AZT was used because this concentration was known to inhibit HIV-1 replication by 90% (IC90). Virus growth was detected in cultures containing 100 nM AZT after day 9. In contrast, the results indicated that no significant amounts of HIV-1 replication was detected in the presence of 25 μM B compound for 15 days.

Experimental Procedures

Cells, transfection, infections, and p24 antigen assay. 293T cells (ATCC) were maintained in the presence of Dulbecco's Modified Eagle's Medium (Cellgro), 10% fetal calf serum (HyClone Laboratories), penicillin (50 U/ml; Gibco) and streptomycin (50 μg/ml; Gibco). 293T cells were plated at a density of 5×10⁶ per 100-mm-diameter dish and transfected using Transfection MBS Mammalian Transfection Kit (Stratgene). Transfected cell supernatants were harvested 48 hours after transfection, clarified, and were used to infect target cells. 293T target cells were plated at a density of 1×10⁵ per 35-mm-diameter dish. Virus containing media was diluted 100-fold and was used to infect 293T cells target cells for 1 hour as previously described, Halvas, E. K. et. al., (2000), Journal of Virology 74:6669-6674. The 293T target cells were incubated with media containing the test compounds for 4 hours prior to infection, 1 hour during infection, and 24 hours post infection.

H9 T-lymphoid cells (ATCC) were maintained in the presence of RPMI 1640 Medium (Cellgro), 20% fetal calf serum (HyClone Laboratories), penicillin (50 U/ml; Gibco) and streptomycin (50 μg/ml; Gibco), 2 μg/ml Polybrene (Sigma). H9 (5×10⁵) target cells were infected using undiluted supernatants containing replication competent HIV-1 virus equivalent to 1000 ng of p24 antigen in 2 ml of medium. After 4 hours of infection, H9 cells were washed, resuspended in 2 ml of cell culture medium containing an appropriate drug and maintained in 35-mm-diameter dish. Cultures were fed every 3 days by removing 1 ml of cell culture suspension and replacing with equivalent amount of fresh medium containing an appropriate drug. At each 3-day interval, cleared supernatants were assayed for p24 antigen, using enzyme-linked immunosorbent assay (ELISA) (p24 core profile kit; DuPont).

Luciferase assay. Infected cells were washed with PBS and lysed in 400 μL of reporter lysis buffer (Promega) 72 hours after infection. Samples were subjected to one freeze-thaw cycle, and cell membranes were removed by centrifugation. Luciferase activity was measured following addition of 100 μL of substrate (Promega) to 20 μL of cell lysate using TD20/20 Luminometer (Promega).

Cell proliferation inhibition assay. The effects of inhibitors on cell toxicity were determined by using Cell Proliferation Kit II (XTT) (Roche Molecular Biochemicals). Target cells (293T) were plated at a density of 1×10³ per well of 96-well plate. Twenty-four hours later cells were treated with an inhibitor for 36 hours. After inhibitor was removed, cells were incubated for additional 48 hours and then were stained with 50 μL of the XTT labeling mix per well (Roche Molecular Biochemicals). Spectrophotometrical absorbance of the samples was measured 4 hours after addition of the labeling mix at wavelengths of 450 nm with a reference point at 700 nm in a 96-well plate reader.

Results

A total of 18 compounds, including 4 compounds of the invention (A-D) were tested for their ability to inhibit HIV-1 infection in a single cycle assay. Three of the 4 compounds (A-C) exhibited significant anti-HIV-activity. In contrast, a structurally related compound lacking the azido groups (E) showed no antiviral activity. Concentrations of the azido group containing compounds needed for 50% inhibition of HIV-I infection (IC50) were determined. The IC50 values for A and B were 1.6 μM and 0.5 μM, respectively. These IC50 values were significantly lower than the IC50 value for the P10 compound, which was previously reported to inhibit HIV-1 integration in both in vitro and in vivo assays. In addition, the B compound strongly inhibited replication competent HIV-1. These results strongly suggest that the azido-group containing compounds may constitute a novel class of HIV-1 integrase inhibitors.
Example 3: Synthesis of Compounds of the Invention

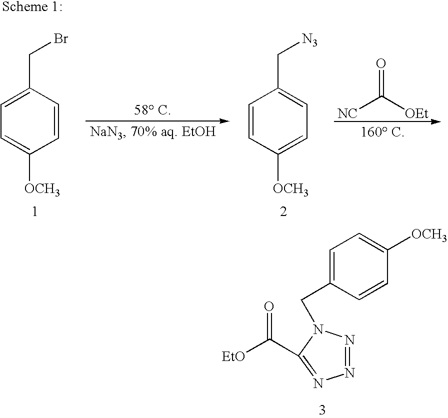

Ethyl 1-(4-methoxybenzyl)-tetrazole-5-carboxylate (3): Prepared according to Lit. report. (Klaubert, D. H.; Sellstedt, J. H.; Guinosso, C. J.; Bell, S. C.; Capetola, R. J. J Med. Chem. 1981, 24, 748-752). white solid (58 %), m.p. 51-53° C., (lit. 50-52° C.) ¹H-NMR (CDCl₃): δ 1.39 (t, J=7.1 Hz, 3H), 4.47 (q, J=7.1 Hz, 2H), 5.80 (s, 2H) 6.81 (d, J=8.8 Hz, 2H), 7.42 (d, J=8.8 Hz, 2H)

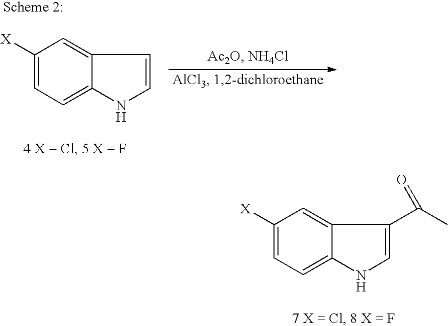

Acylindoles 7 and 8 were synthesized by the Friedel-Crafts type reaction of the corresponding indoles

Ammonium chloride (4 mole equiv) was suspended in ethylene dichloride. Acetic anhydride (2 mole equiv.) was added dropwise via syringe to this suspension at room temperature. After stirring for 15 min, indole derivative (1 mole equiv) in ethylene dichloride was added dropwise and stirring was continued for 2 h. AlCl₃ (2 mole equiv) was added to this colorless heterogeneous mixture. The mixture becomes homogeneous. Then 1 mole equiv. of acetic anhydride was added and stirred for 30 more minutes. Poured into crushed ice. Extracted with ethyl acetate. Washed with water, sat aq. NaHCO₃, dried and concentrated under reduced pressure. The residue was recrystalized from ethyl acetate.

3-Acetyl-5-chloroindole (7): white solid, 75 %; ¹H-NMR (DMSO-d₆): 2.45 (s, 3H), 7.22 (dd, J=8.1, 2.2 Hz, 1H), 7.49 (d, J=8.8 Hz, 1H), 8.14 (s, 1H), 8.38 (d, J=2 Hz, 1H), 12.10 (brs, 1H); mass: m/z 194 (MH+, 100)

3-Acetyl-5-fluoroindole (8): Above procedure was used: colorless solid; 73 %;, m.p. 200-201° C. (lit. 200-201.5° C.; Ketcha, D. M.; Gribble, G. W.; J Org. Chem. 1985, 50, 5451-5457) ¹H-NMR (DMSO-d₆): δ 2.48 (s, 3H), 7.11 (td, J=6.1, 2.4 Hz, 1H), 7.52 (dd, J=4.6, 4.2 Hz, 1H), 7.86 (dd, J=10, 2.2 Hz, 1H), 8.42 (d, J=2.9 Hz, 1H), 12.06 (brs, 1H); mass: m/z (%) 178 (MH+, 100), 162 (16.5)

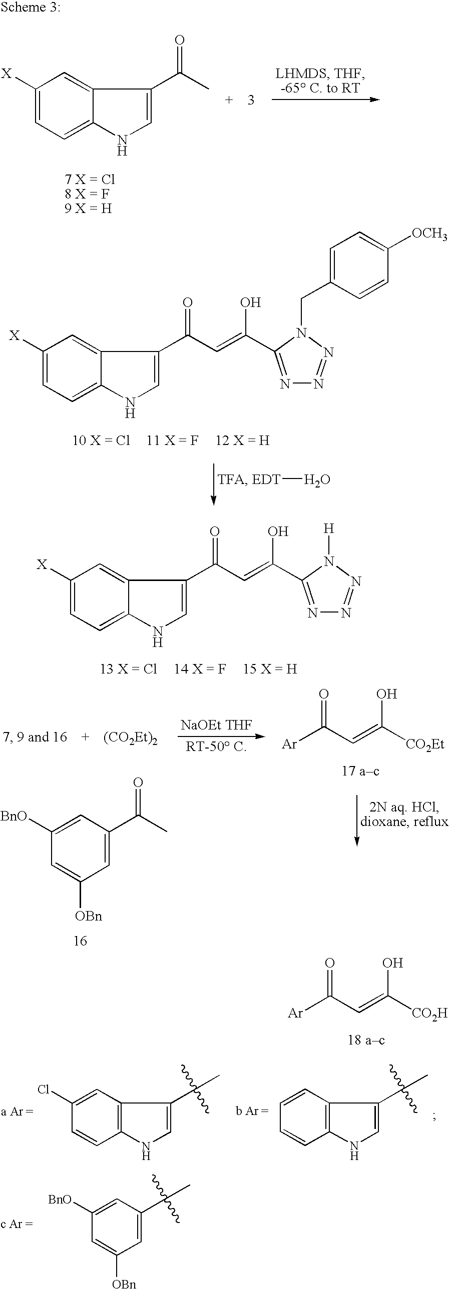

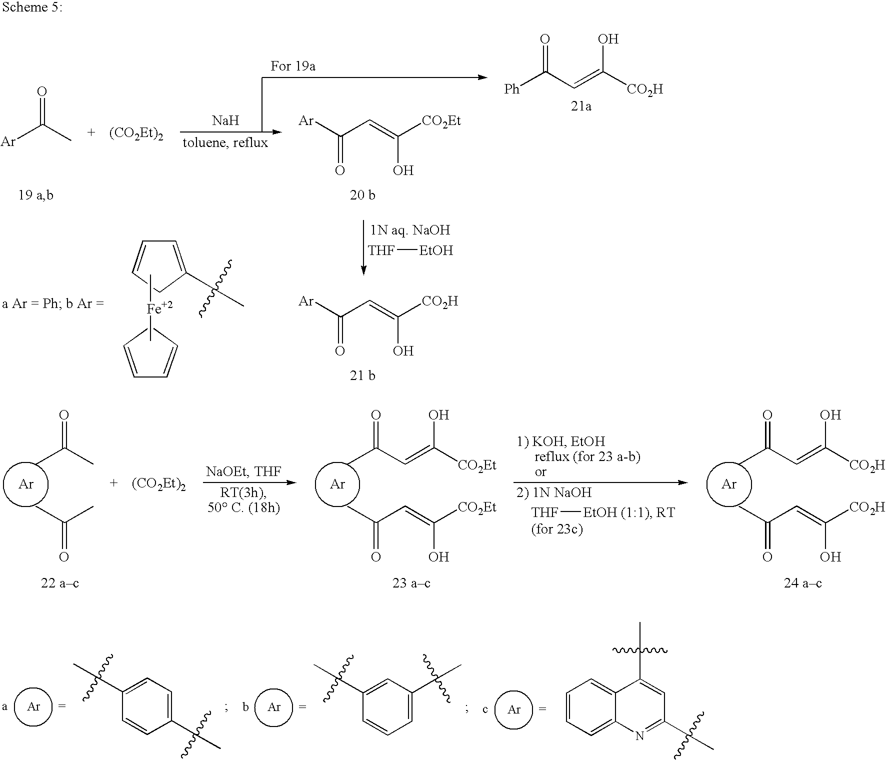

Procedure A: To a stirred solution of KHMDS or LHMDS (1.0 M in THF, 3.0 mole equivalent) at −78° C. aryl ketone in anhydrous THF was added dropwise. After the addition is complete, the solution was stirred at −20° C. for 2 h. Then the reaction mixture was recooled to −78° C. and a solution of 3 in THF was added dropwise. The mixture was allowed to warm to room temperature over one hour and stirred at RT for 2 h. Quenched by the addition of sat. aq. NH₄Cl solution. The two layers were separated and the aqueous layer was extracted with ethyl acetate. The combined organic layers were washed with water, dried and concentrated under reduced pressure. Ethyl acetate was added to the residue. Bright yellow solid separates, which was recrystalized to get the pure compound.

Procedure A-1: General procedure for the deprotection of PMB group: A mixture of compound, TFA, ethylene dithiol -water (1:1) was stirred at RT for 20 h. The volatiles were removed under reduced pressure. The residue was triturated with ether, supernatant liquid was removed. The yellow solid was re-suspended in ether (2 times) and supernatant liquid was removed. Washed with water, EtOAc and dried to get pure product.

Procedure B: To a stirred solution of NaOEt in anhyd. THF at room temperature were added in succession diethyl oxalate in THF and aryl ketone in THF dropwise. The resulting orange yellow mixture was stirred at room temperature for 3 h and at 50 ° C. for 18 h. The solvent was removed under reduced pressure and the residue was washed with ether and filtered. The yellow solid was washed with 1N aq. HCl and water, dried to get the desired diketo acid. Pure product was obtained by recrystallization.

Procedure C: Aryl ketone (1 mole equiv) was dissolved in toluene. NaH was added (2.0 mole equiv) and stirred for 10 min. Finally, 1.5 mole equiv. of diethyl oxalate was added dropwise and the resulting mixture was refluxed for 1 h. Black solid separates. Cooled to RT, filtered, residue was washed with diethyl ether, acidified with 1N hydrochloric acid, (a) washed with water, dried to get the required compound, (b) aqueous layer was extracted with ethyl acetate. The organic layer was washed with water, dried and concentrated under reduced pressure. The pure product was obtained by column chromatographic purification of the crude product thus obtained.

Procedure C2

General procedure for hydrolysis of the ethyl ester:

(a) The ester was dissolved in dioxane and 1N aq. HCl was added. The resulting solution was refluxed for 4 h. The solvent was removed under reduced pressure. Water was added to the residue. Yellow solid separates. It was filtered, washed with water and dried.

1) The ester (1 mole equiv.) was added to mixture of KOH (3.8 mole equiv) in ethanol and refluxed overnight. After the reaction mixture was cooled to RT, it was diluted with water and acidified to pH 3 with 3N aq. HCl. The resulting precipitate was collected by filtration and dried to get pure acid. (Flynn, D. L.; Belliotti, T. R.; Boctor, A. M.; Connor, D. T.; Kostlan, C. R.; Nies, D. E.; Ortwine, D.F.; Schrier, D. J.; Sircar, J. C. J Med. Chem. 1991, 34, 518-525)

2) The ester (1 mole equiv) was dissolved in 1:1 THF/EtOH and treated with 1N aq. NaOH (5 mole equiv.) and stirred 1 h. The reaction mixture was extracted twice with diethyl ether, then acidified to pH1-2 with 2N HCl and extracted repeatedly with EtOAc. The organic layers were combined, washed with water, brine and dried, concentrated and purified.

1-(5-Chloroindol-3yl)-3-hydroxy-3-[1-(4-methoxybenzyl)tetrazol-5-yl)propenone (10): Procedure A was used. After recrystallization from EtOAc afforded yellow solid (84%); ¹H-NMR (400 MHz, DMSO-d₆): 3.72 (s. 3H), 5.94 (s, 2H), 6.96 (d, J=7.3 Hz, 2H), 7.28 (s, 1H), 7.36 (d, J=8.8 Hz, 3H), 7.53 (d, J=8.1 Hz, 1H), 8.25 (s, 1H), 8.58 (s, 1H), 12.38 (brs, 1H); mass: m/z (%) 410 (M+H, 18.6), 121 (100); (−ve FAB) HRMS: calcd. for C₂H₁₅CIN₅O₃(M−H): 408.0863 found 408.0878.

1-(5-Fluoroindol-3-yl-3-hydroxy-3-[1-(4-methoxylbenzyl)-tetrazol-5-yl)-propenone (11): General procedure A was followed for the coupling yellow solid (60%); ¹H-NMR (DMSO-D₆): δ 3.76 (s, 3H), 5.97 (s, 2H), 6.97 (d, J=8.3 Hz, 2H), 7.19 (t, J=9.5 Hz, 1H), 7.28 (s, 1H), 7.38 (d, J=8.5 Hz, 2H), 7.58 (dd, J=3.4, 4.4 Hz, 1H), 7.90 (d, J=8.8 Hz, 1H), 8.89 (s, 1H), 12.71 (brs, 1H); mass: m/z (%) 394 (MH⁺, 12.1), 162 (14.8), 121 (100); (−ve FAB) HRMS: calcd. for C₂₀H₁₅FN₅O₃ (M−H): 392.1159 found 392.1143

1-(Indol-3-yl)-3-hydroxy-3-[1-(4-methoxybenzyl)-tetrazol-5-yl]-propenone (12): Procedure A was followed. Crude product was recrystalized from EtOAc to get yellow solid (88 %). ¹H-NMR (DMSO-d₆): δ 3.69 (s, 3H), 6.88 (d, J=10.3 Hz, 2H), 7.20 (s, 1H), 7.20-7.27 (m, 2H), 7.28 (d, J=10.5 Hz, 2H), 7.45-7.51 (m, 1H), 8.09-8.15 (m, 1H), 8.64 (d, J=5.2 Hz, 1H); mass :m/z 376 (MH⁺, 14.5), 121 (100); Anal: calc. C 63.99. H 4.57, N 18.66; found. C 64.04, H 4.54, N 18.55; (−ve FAB) HRMS calcd. for C₂₀H₁₆N₅O₃ 374.1253, found 374.1248 .

1-(5-Chloroindol-3-yl)-3-hydroxy-3-(1 H-tetrazol-5-yl)-propenone (13): Yellow solid (70%) ,mp. 250° C.; ¹H-NMR (DMSO-d₆): δ 7.26 (s, 1H), 7.32 (dd, J=8.7, 2.1 Hz, 1H), 7.56 (d, J=8.7 Hz, 1H), 8.21 (d, J=2.1 Hz, 1H), 8.84 (d, J=3.3 Hz, 1H), 12.6 (brs, 1H); mass (−ve FAB, m/z)): 288 (M−H)⁺(100%)

1-(5-Fluoroindol-3-yl)-3-hydroxy-3-(1H-tetrazol-5-yl)-propenone (14): yellow solid (86 %), ¹H-NMR (DMSO-d₆): δ 7.15-7.31 (m, 2H), 7.55-7.58 (m, 1H), 7.89-7.99 (m, 1H), 8.89 (t, J=3.2 Hz, 1H), 12.63 (brs, 1H); Mass: m/z, (%) 274 (MH⁺, 22.9), 121 (100).

1-(Indol-3-yl)-3-hydroxy-3-(1 H-tetrazol-5-yl)-propenone (15): yellow solid, recrystallized from DMF (75 %); ¹H-NMR (400 MHz, DMSO-d₆): δ 7.25 (s, 1H), 7.26-7.31 (m, 2H), 7.51-7.55 (m, 1H), 8.22 (dd, J=5.9, 2.9 Hz, 1H), 8.76 (d, J=3.7 Hz, 1H), 12.45 (s, 1H); Anal. calc. for C₁₂H₉N₅O₂0.86C₃H₇NO: C 55.00, H 4.72, N 25.80, found C 55.00, H 4.88, N 25.55.

Ethyl 4-(5-chloroindol-3-yl)-2-hydroxy-4-oxo-2-butenoate (17a): Prepared according to the procedure B. Recrystallized from dioxane to get (82%) as yellow solid; mp. 219-225° C.; ¹H-NMR (DMSO-d₆): δ 1.36 (t, J=7.1 Hz, 3H), 4.35 (q, J=7.1 Hz, 2H), 7.08 (s, 1H), 7.35 (d, J=8.5 Hz, 1 H), 7.75 (d, J=8.6 Hz, 1H), 8.25 (s, 1H), 8.87 (d, J=1.2 Hz, 1H), 12.67 (brs, 1H); mass: m/z 294 (MH⁺, 29.5), 220 (16.5); Ref Chem. Heterocycl. Compd. 1973, 9, 1374.

Ethyl 4-(indol-3-yl)-2-hydroxy-4-oxo-2-butenoate (17b): Procedure B was followed ; thus 3-acetyl indole (0.5 g, 3.14 mmol) was treated with diethyl oxalate (0.85 ml, 6.28 mmol) in presence of NaOEt (0.43 g, 6.37 mmol) to get the brown solid, which was recrystallized from methanol-hexane to get the desired compound as yellow solid (80%); mp.>300° C.; ¹H-NMR (DMSO-d₆): δ 1.35 (t, J=7.1 Hz, 3H), 4.31 (q, J=7.1 Hz, 2H), 6.97 (s, 1H), 7.28 (t, J=3.6 Hz, 2H), 7.53 (d, J=6.4 Hz, 1H), 8.28 (d, J =7.1 Hz, 1H), 8.67 (s, 1H); mass, m/z : 260 (MH⁺, 100), 186 (69.4), 144 (28.5) ; Anal.: calc. for C₁₄H₁₃NO₄ C 64.86, H 5.05, N 5.40; found C 64.54, H 5.16, N 5.44 ref J. Chem. Soc., 1957, 4810, 4813

Ethyl 4-[(3,5-dibenzyloxy)phen-1-yl]-2-hydroxy-4-oxo-2-butenoate (17c): Procedure C was followed. 5.0 g of the ketone gave 5.52 g (85%) of the product after recrystallization from EtOAc-Hexane as bright yellow solid; m.p. 77-78° C.; ¹H-NMR (CDCl₃): δ 1.51 (t, J=7.1 Hz, 3H), 4.49 (q, J=7.1 Hz, 2H), 5.18 (s, 4H), 6.95 (s, 1H), 7.11 (s, 1H), 7.33 (s, 2H), 7.35-7.59 (m, 10 H); mass: 433 MH⁺, 4.0), 317 (4.0), 91 (100); Anal.calc. (C₂₆H₂₄O₆. 0.026 EtOAc) C 72.05, H 5.56 ; found C 72.05, H 5.65.

4-[5-Chloroindol-3-yl]-2-hydroxy-4-oxo-2-butenoic acid (18a): Hydrolyzed according to procedure(a):Residue was washed with water and dioxane and dried to get the pure product as brown solid (65%); 220-225° C.; ¹H-NMR (DMSO-d₆): δ 7.06 (s, 1H), 7.29 (dd, J=8.7 Hz, 2.4 Hz, 1H), 7.53 (d, J=8.7 Hz, 1H), 8.21 (d, J=2.4 Hz, 1H), 8.77 (d, J=3.6 Hz, 1H), 12.5 (brm, 1H)

4-(Indol-3-yl)-2-hydroxy-4-oxo-2-butenoic acid (18b): Hydrolyzed according to the procedure (a): thus 0.1 g (0.39 mmol) was refluxed with 1.0 ml of 1N aq. HCl in 10 ml of dioxane. Recystallization from ethyl acetate, after usual work-up, gave 0.05 g of yellow solid (56 %); mp. 202-210° C.; ¹H-NMR (DMSO-d₆): δ 7.06 (s, 1H), 7.30 (t, J=3.6 Hz, 2H), 7.53 (s, 1H), 8.27 (s, 1H), 8.75 (d, J=2.7 Hz, 1H), 12.44 (s, 1H); Mass: (−ve FAB) (m/z): 230 (M−H, 100), 158 (13.9)

4- [(3,5-Dibenzyloxy)phen-1-yl]-2-hydroxy-4-oxo-2-butenoic acid (18c): Hydrolyzed according to Proc.(a), recrystallized from EtOAc to get yellow solid (75%); m.p.: 170-172° C.; ¹H-NMR (CD₃OD): δ 5.19 (s, 4H), 6.96 (s, 1H), 7.09 (s, 1H), 7.29 (s, 2H), 7.35-7.61 (m, 10H); mass: (−ve FAB) m/z (%): 404 (M^{+bl , 44.9}), 403 (M−H, 100), 331 (34.7); Anal. calc. for (C₂₄H₂₀O₆. 0.15 EtOAc) C 70.68, H 5.07; found C 70.68, H 5.02

Ethyl 4-(2-ferrocenyl)-2-hydroxy-4-oxo-2-butenoate (20b): Procedure C was followed; 0.2 g (0.877 mmol) of acetyl ferrocene was coupled with 0.18 ml (1.315 mmol) of diethyl oxalate in presence of 0.042 g of NaH (1.753 mmol) to get 140 mg of the pure compound after column (20% EtOAc in Hexane as eluent ) as violet solid, m.p: 66-69° C.; ¹H-NMR (CDCl₃): δ 1.42 (t, J=7.1 Hz, 3H), 4.26 (s, 5H), 4.39 (q, J=7.1 Hz, 2H), 4.68 (s, 2H), 4.92 (s, 2H), 6.56 (s, 1H); mass (FAB): m/z (%) 328 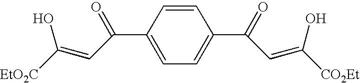
(100, M¹), 254 (23.8).

General procedure B was used with slight modification: Thus, 1.0 g (6.17 mmol) of the diketone (22a) was coupled with 3.60 g (24.68 mmol) of diethyl oxalate using 1.70 g (25.03 mmol) of NaH in 90 ml Of THF to get an yellow residue after usual work-up. It was suspended in 1N aq. HCl for 15 min, filtered, washed with water, dried and recrystallized from EtOAc to get 1.83 g (82 %) of 32a as shiny yellow solid: m.p.: 113-115° C.; ¹H-NMR (DMSO-d₆): δ 1.37 (t, J=7.1 Hz, 6H), 4.37 (q, J=7.1 Hz, 4H), 7.19 (s, 2H), 8.25 (s, 4H); mass: m/z (%) 363 (MH⁺, 100), 289 (49.6), 247 (18.9); Anal: calc. C 59.67, H 5.01: found C 59.54, H 5.07; (−ve FAB) HRMS: calcd. for C₁₈H₁₈O₈ 362.1002 found 362.1007 ref. El-Bahaie, S.; Assy, M. G.; Hassanien, M. M. J. Ind. Chem. Soc. 1990, 67, 757-758.

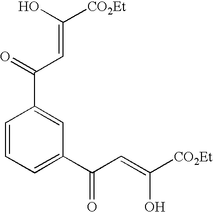

General procedure B with modification was used as described above.: 0.5 g (3.08 mmol) of the diketone 22b gave 0.89 g (80%) of the pure product 32b after recrystallization from EtOAc as yellow solid; m.p.: 93-95° C.; ¹H-NMR (DMSO-d₆): δ 1.37 (t, J=7.1 Hz, 6H), 4.39 (q, J=7.1 Hz, 4H), 7.21 (s, 2H), 7.81 (t, J=7.8 Hz, 1H), 8.38 (d, J=7.6 Hz, 2H), 8.59 (s, 1H); mass (+ve FAB): m/z 363 (MH⁺, 100), 289 (27.4), 247 (24.6), 215 (35.4), 173 (42.6); Anal. calc. for (C₁₈H₁₈O₈ 0.22 EtOAc) C 59.35, H 5.17; found C 59.35, H 5.10.

4-Phenyl-2-hydroxy-4-oxo-2-butenoic acid (21a): 1.0 g (8.33 mmol) of acetophenone in 20 ml of toluene was coupled with diethyl oxalate (1.7 ml, 12.5 mmol) in presence of 0.40 g (16.6 mmol) of NaH according to procedure C. The orange yellow oil obtained after the work-up was recrystallized from diethyl ether-hexane hexane to get 150 mg of the acid as yellow solid; m.p. 155-158° C. ¹H-NMR.(CDCl₃): 7.15 (s, 1H), 7.53 (t, J=7.3 Hz, 2H), 7.65 (t,=7.3 Hz, 1H), 8.00 (d, J=7.1 Hz, 2H); mass (−ve FAB): m/z 191 (M−H, 100), 119 (28.8)(ref. Andreichikov, Y. S.; Maslivets, A. N.; Smimova, L. I.; Krasnykh, O. P.; Kozlov, A. P.; Perevozchikov, L. A. J Org. Chem. USSR, 1987, 23, 1378-1387)

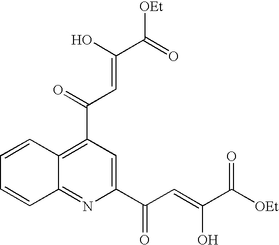

Gen. Proc. C was followed; brown solid (77 %). ¹H-NMR (DMSO-d₆): 1.22 (t, J=7.3 Hz, 3H), 1.33 (t, J=7.3 Hz, 3H), 4.11 (q, J=6.6 Hz, 2H), 4.32 (q, J=6.6 Hz, 2H), 7.48 (s, 1H), 7.73 (t, J=7.3 Hz, 1H), 7.88 (t, J=7.3 Hz, 1H), 8.05 (s, 1H), 8.21 (d, J=8.8 Hz, 1H), 8.32 (d, J=8.1 Hz, 1H).; mass: m/z 414 (MH⁺, 100)

4-(2-ferrocenyl)-2-hydroxy-4-oxo-butanoic acid (21b): Gen. Procedure (c) was used for the hydrolysis. Purified by flash chromatography (10% MeOH in Chloroform) to get 25b as violet solid; mp. 160-163° C.; ¹H-NMR (CDCl₃): δ 4.23 (s, 5H), 4.69 (s, 2H), 4.92 (s, 2H), 7.27 (s, 1H); mass (−ve FAB): m/z (%) 299 (M−H, 100), 227 (42.6); HRMS calcd for C₁₄H₁₁FeO₄ (M−H) 299.00067, found 299.00158

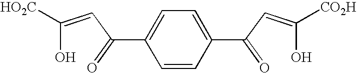

Gen. Procedure (b) was used with slight modification : a mixture of 0.1 g (0.276 mmol) of diester 23a and 0.117 g (2.09 mmol) of KOH in 6 ml of EtOH was refluxed for 24 h. Cooled, diluted with water (5 ml) and acidified with 3N aq. HCl. Yellow solid separates, which was filtered, washed with ether. It was suspended in 3N aq. HCl for 15 min, washed with water and dried and recrystallized from EtOAc to get 0.066 g (78%) of diacid 24a as yellow solid, ¹H-NMR ( DMSO-d₆): δ 7.17 (s, 2H), 8.23 (s, 4H); mass (−ve FAB): m/z 306 (M.−, 100), 233 (65.0).

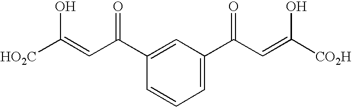

General procedure (b) with slight modification was used as described above: Thus 0.1 g (0.276 nimol) of the diester 23b gave 0.064 g (75%) of the diacid 24b as yellow solid, ¹H-NMR (DMSO-d₆): δ 7.01 (s, 2H), 7.76 (t, J=8.1 Hz, 1H), 8.31 (d, J=7.6 Hz, 2H), 8.56 (s, 1H); mass (−ve FAB): m/z 305, (M−H, 100), 233 (22.8); (−ve FAB) HRMS calcd. for C₁₄H₉O₈ 305.0297 found 305.0303.

2,4-Diacetylquinoline (22c): White solid, mp. 66-68° C.; ¹H-NMR (CDCl₃): δ 2.80 (s, 3H), 2.90 (s, 3H), 7.72-7.88 (m, 2H), 8.27 (dd, J=7.3, 1.2 Hz, 1H), 8.39 (s, 1H), 8.57 (dd, J=6.8, 0.5 Hz, 1H); mass, m/z: 214 (MH⁺, 100) (24c): Gen. Proc. c was used for hydrolysis. Brown solid, purified by HPLC (65%). ¹H-NMR (DMSO-d₆): 6.69 (s, 2H), 7.85 (t, J=8.1 Hz, 1H), 7.97 (t, J=6.6 Hz, 1H) 8.13 (s, 1H), 8.29 (t, J=8.1 Hz, 2H); Anal. calc. for C₁₇H₁₁NO₈.0.84H₂O C 54.79, H 3.40, N 3.94, found C 54.79, H 3.41, N 3.76.

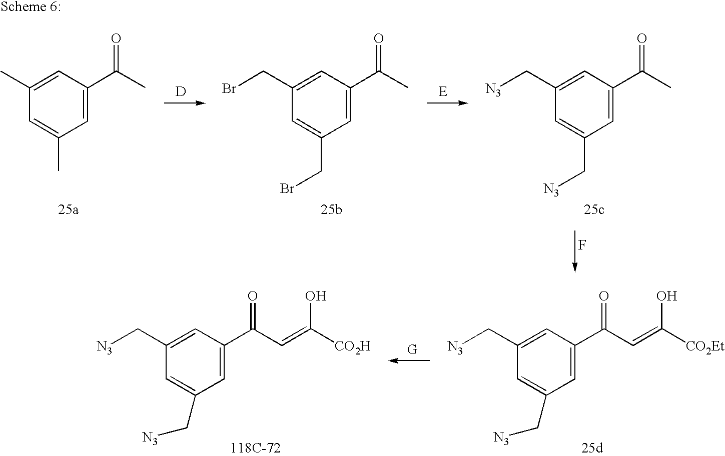

General procedure D:

The mixture of 3′, 5′-dimethylacetophenone(1.02 g, 6.89 mmol), NBS(2.45 g, 13.78 mmol) and benzoyl peroxide(16.7 mg, 0.07 mmol) in CCl₄ (15 ml) was refluxed for 3 hours. The solid was removed by filtration and the solvent was evaporated under reduced pressure. The residue was purified by silica gel flash column chromatography(hexanes:ethyl acetate=5:1) to provide 3′, 5′-dibromomethylacetophenone 25b (720 mg, 34%). mp: 87-88° C. ¹H NMR(CDCl₃) δ7.88(s, 2H), 7.62(s, 1H), 4.50(s, 4H), 2.61(s, 3H). FAB-MS (⁺VE) m/z: 305(MH⁺). C₁₀H₁₀Br₂O Calcd: C, 39.25; H, 3.29. Found: C, 39.24; H, 3.31.

Compound 25c:

General procedure E:

The mixture of 25b (700 mg, 2.29 mmol) with sodium azide (595 mg, 9.15 mmol) in acetone-H₂O (5:1) (30 ml) was refluxed for 3 h, then concentrated, and diluted (H₂O). The aqueous phase was extracted with ether (3×30ml). The organic layer was dried with Na₂SO₄ and taken to dryness. The residue was chromatographed (hexanes: ethyl acetate=3:1) to afford 3′, 5′-diazidomethylacetophenone (25c) (520 mg, 99%). ¹H NMR(CDCl₃) δ 7.86(s, 2H), 7.49(s, 1H), 7.45(s, 4H), 2.63(s, 3H). FAB-MS (+VE) m/z: 231(M⁺).

Compound 25d:

General procedure F:

To an oven dried 100 ml flask fitted with argon inlet was added 3′, 5′-diazidomethylacetophenone (25c) (162 mg, 0.704 mmol) in toluene (4 ml), sodium hydride (34 mg, 1.41 mmol) was added followed by diethyl oxalate (143 μl, 1.06 mmol) at room temperature following by stirring at 60° C. for 3 h. The reaction was quenched with H₂O and the mixture was acidified to PH=6 with 2N HCl and extracted with ethyl acetate(30 mlx4). The organic layer was dried over Na₂SO₄ and taken to dryness. The residue was purified by silica gel chromatography (hexanes:ethyl acetate=1:1) to provide product (25d) (169 mg, 73%). ¹H NMR (CDCl₃) δ7.90(s, 2H), 7.53(s, 1H), 7.08(s, 1H), 4.49(s, 4H), 4.42(q, 2H), 1.43(t, 3H). FAB-MS (⁺VE) m/z: 331(MH⁺).

Compound A:

General procedure G:

A solution of compound 25d (25 mg, 0.09 mmol) in dioxane (3 ml) was treated with 1N NaOH (3 ml) and stirred 1 h. The mixture was acidified with 2 N HCl to PH=2-3 and taken to dryness. The residue was purified by preparative HPLC to afford compound A (18 mg, 80%). mp:>300° C. ¹H NMR (DMSO-d₆) δ 7.94(s, 2H), 7.54(s, 1H), 7.00(s, 1H), 4.62(s, 4H). MS: 176(13), 154(100). 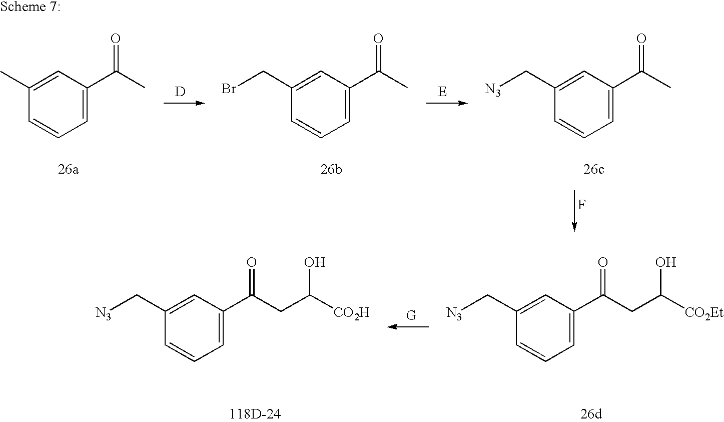
Compound 26b:

Follow the general procedure D.

Compound 26c:

Follow the general procedure E. Yield:>99%. ¹H NMR (CDCl₃): δ7.91-7.87(m, 2H), 7.53-7.46(m, 2H), 4.40(s, 2H), 2.60(s, 3H). FAB-MS (⁺VE) m/z: 176(MH⁺).

Compound 26d:

Follow the general procedure F.

Yield: 78%. ¹H NMR (CDCl3): δ7.95-7.92(m, 2 H), 7.56-7.49(m, 2H), 7.05(s, 1H), 4.43(s, 2H), 4.39(q, 2H), 1.40(t, 3H). FAB-MS (⁺VE) m/z: 276(MH⁺). C₁₃H₁₃N₃O₄ Calcd: C, 56.72; H, 4.76; N, 15.27. Found: C, 57.12; H, 4.71; N, 15.04.

Compound B:

Follow the general procedure G except that the product was obtained by collecting the solid followed by washing with H₂O and hexanes.

Yield: 26%. ¹H NMR (DMSO-d₆): δ8.07(s, 1H), 8.03(d, J=8 Hz, 1H), 7.68(d, J=8 Hz, 1H), 7.60(t, J=7 Hz, 1H), 7.06(s, 1H), 4.59(s, 2H). FAB-MS (⁻VE) m/z: 246(M-H⁺). 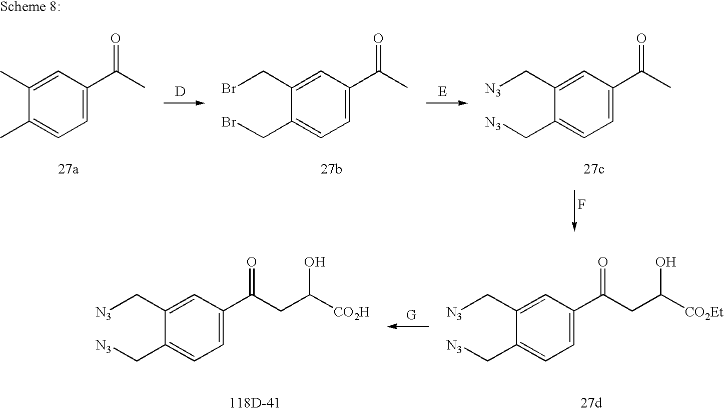
Compound 27b:

Follow the general procedure D.

Yield: 34%. ¹H NMR (CDCl₃): δ 7.92(d, J=1.6 Hz, 1H), 7.84(dd, J=8 Hz, 1.6 Hz, 1H), 7.45(d, J=8 Hz, 1H), 4.65(s, 2H), 4.63(s, 2H), 2.58(s, 3H). FAB-MS (⁺VE) m/z: 305(MH⁺). C₁₀H₁₀Br₂O. 0.38 H₂O Calcd: C, 40.16; H, 3.46. Found: 40.16; H, 3.35.

Compound 27c:

Follow the general procedure E.

Yield: 45%. ¹H NMR (CDCl₃): δ7.94-7.91(m, 2H), 7.47(d, J=8 Hz, 1H), 4.47(s, 2H), 4.46(s, 2H), 2.60(s, 3H). FAB-MS (⁺VE) m/z: 231(MH⁺). C₁₀H₁₀N₆O Calcd: C, 52.17; H, 4.37; N, 36.50. Found: C, 52.45; H, 4.30; N, 36.36.

Compound 27d:

Follow the general procedure F except that the reaction was kept at 0° C. to room temperature overnight.

Yield: 77%. ¹H NMR (CDCl₃): δ7.96-7.94(m, 2H), 7.50(d, J=8.5 Hz, 1H), 7.03(s, 1H), 4.48(s, 4H), 4.37(q, 2H), 1.38(t, 3H). FAB-MS (⁺VE) m/z: 331(MH⁺).

Compound C:

Follow the general procedure G.

Yield: 55%. ¹H NMR (DMSO-d₆): δ8.09(s, 1H), 8.04(m, 1H), 7.61(d, J=8 Hz, 1H), 4.66(s, 4H). FAB-MS (−VE) m/z: 301(M−H⁺). 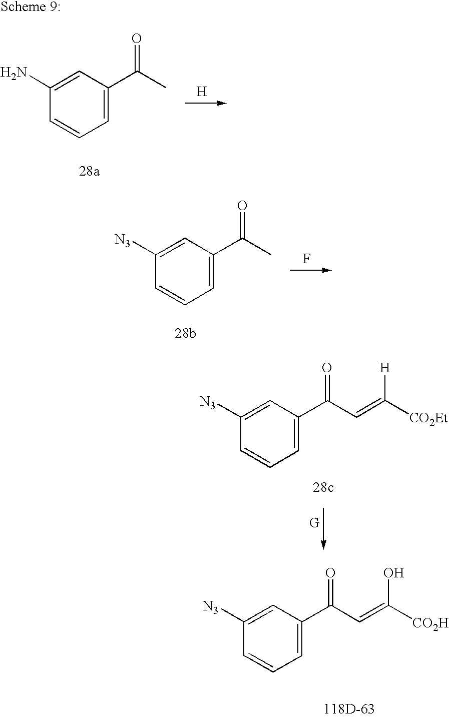
Compound 28b:

Procedure H. (Bull. Chem. Soc. Jpn., 1986, 2317-2320).

3′-aminoacetophenone (540 mg, 4 mmol) (28a) was dissolved in acetone (55 ml) in the dark. The mixture was cooled to 0° C., and HCl (0.22 M, 55 ml) was added. After 10 min of stirring under a nitrogen atmosphere, NaNO₂ (1.21 g, 17.6 mmol) was added, followed 30 min later by NaN₃ (3.25 g, 50 mmol) while maintaining the reaction mixture at 0° C. Fifteen minutes later, ether (30 ml) was added as an overlay. Water was added 45 min later, and the product was isolated by extraction with ether. The organic layer was washed with brine and dried over Na₂SO₄. The solvent was removed under reduced pressure and purified by flash chromatography (hexanes: ethyl acetate=4: 1) to afford the product (536 mg, 83%).

¹; H NMR (CDCl₃): δ7.67(m, 1H), 7.57(m, 1H), 7.41(t, J=8 Hz, 1H), 7.15(m, 1H), 2.57(s, 3H). FAB-MS (⁺VE) m/z: 162(MH⁺).

Compound 28c:

Follow the general procedure F.

Yield:>99%. ¹H NMR (CDCl₃): δ7.70(m, 1H), 7.60(t, J=3 Hz, 1H), 7.32(t, J=8 Hz, 1H), 7.22(m, 1H), 7.00(s, 1H), 4.37(q, 2H), 1.38(t, 3H). FAB-MS (⁺VE) m/z: 262(MH⁺).

Compound D:

Follow the general procedure G.

Yield: 70%. ¹H NMR (DMSO-d₆): δ7.87(d, J=8 Hz, 1H), 7.68(t, J=2 Hz, 1H), 7.59(t, J=7.8 Hz, 1H), 7.43(m, 1H), 7.09(s, 1H). FAB-MS (⁻VE) m/z: 232(M−H⁺). 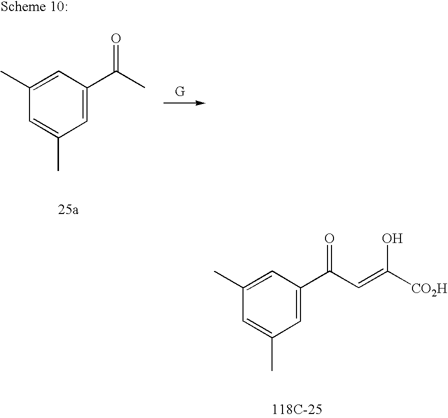
Compound E:

Follow the general procedure F. The beta-diketo acid analogue E was obtained as major product.

Yield: 68%. Mp: 137° C.(dec). ¹H NMR (DMSO-d₆) δ 7.53(s, 2H), 7.19(s, 1H), 2.32(s, 6H). FAB-MS (−VE) m/z: 219(M−H)⁺. C₁₂H₁₂O₄. 0.128H₂O Calcd: C, 64.71; H, 5.56. Found: C, 64.71. H, 5.65. 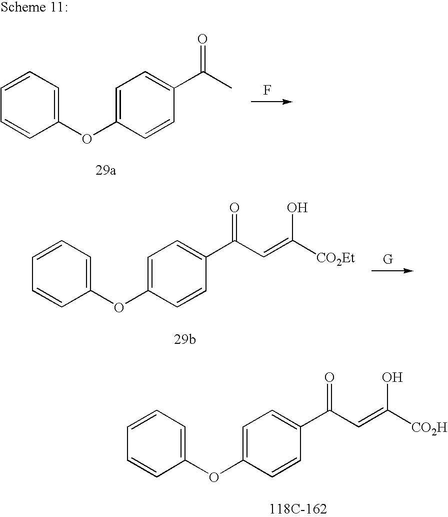
Compound 29b:

Follow the general procedure F.

Yield: 83%. ¹H NMR (CDCl₃) δ 8.05-7.97(m, 2H), 7.44-7.40(t, J=7.7 Hz, 2H), 7.24-7.21(m, 2H), 7.11-7.02(m, 4H), 4.40(q, 2H), 1.42(t, 3H). FAB-MS (⁺VE) m/z: 313(MH⁺).

Compound F:

Follow the general procedure G.

Yield: 61%. ¹H NMR (DMSO-d₆) δ 8.09(d, J=8.8 Hz, 2H), 7.48(t, J=8.8 Hz, 2H), 7.27(t, J=7 Hz, 1H), 7.16(d, J=8 Hz, 2H), 7.08-7.03(m, 3H). FAB-MS (⁻VE) m/z: 283(M−H⁺). C₁₆H₁₂O₅. 0.22 H₂O Calcd: C, 66.65; H, 4.35. Found: C, 66.65; H, 4.30 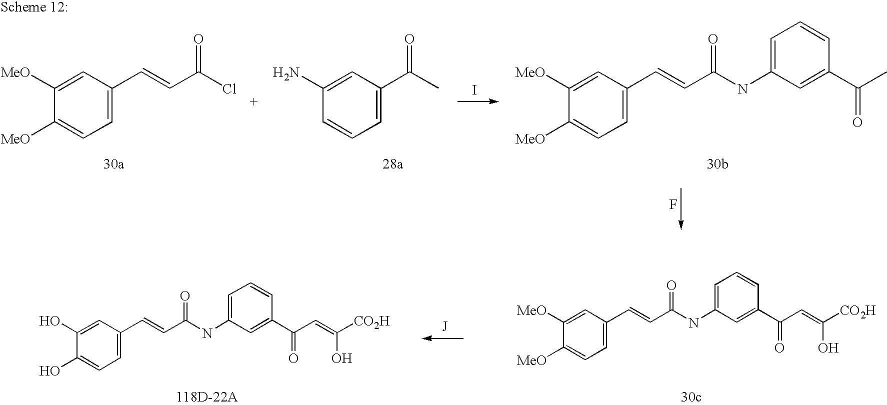
Compound 30b:

Procedure I.

To a mixture of 3,4-dimethylcaffeoic acid (416 mg, 2 mmol) in benzene (8 ml) was added thionyl chloride (730 μl, 10 mmol) and the mixture was refluxed 3 h. After removal of solvent, the residue was added to the mixture of 28a (243 mg, 1.8 mmol) and DMAP (22 mg) in pyridine (8 ml). The resulted mixture was stirred at room temperature overnight. The solvent was removed and the residue was purified by silica gel flash chromatography (hexanes: ethyl acetate=1: 1) to give pure product 30b (377 mg, 56%).

¹H NMR (DMSO-d₆): δ9.94(s, 1H), 9.55(dd, J=8 Hz, 2 Hz, 1H), 9.19(d, J=7.6Hz, 1H), 9.05(d, J=16 Hz, 1H), 8.96(t, J=8 Hz, 1H), 8.63-8.59(m, 2H), 8.38(d, 1H), 7.98(d, J=16 Hz, 1H), 4.39(s, 3H), 4.36(s, 3H), 2.83(s, 3H). FAB-MS (⁺VE) m/z: 326(MH⁺). C₁₉H₁₉NO₄ Calcd: C, 70.13; H, 5.89; N, 4.30. Found: C, 69.99; H, 6.03; N, 4.26.

Compound 30c:

Follow the general procedure F. The beta-diketo acid analogue was obtained as major product.

Yield: 78%. ¹H NMR (DMSO-d₆): δ10.39(s, 1H), 8.40(s, 1H), 8.00(dd, J=8 Hz, 1 Hz, 1H), 7.78(d, J=8 Hz, 1H), 7.59(s, 1H), 7.54(t, J=8 Hz, 1H), 7.23-7.19(m, 2H), 7.04-7.01(m, 2H), 6.68(d, J=16 Hz, 1H), 3.83(s, 3H), 3.80(s, 3H). FAB-MS (−VE) m/z: 396(M−H⁺). C₂₁H₁₉NO₇. 2.1 H₂O Calcd: C, 57.95; H, 5.36; N, 3.21. Found: C, 57.95; H, 5.37; N, 3.16.

Compound G:

Procedure J.

The solution of compound 30c (40 mg, 0.1 mmol) in CH₂Cl, (5 ml) was cooled to 0° C. followed by addition of BBr₃ (1.0 M in CH₂Cl₂, 0.5 ml) the mixture was stirred at 0° C. to room temperature for 4 h. The solvent was removed under reduced pressure and the residue was purified by preparative HPLC (10 mg, 27%).

Yield: 27%. ¹H NMR (DMSO-d₆): δ10.33(s, 1H), 9.47(s, 1H), 9.20(s, 1H), 8.37(s, 1H), 7.98(d, J=8 Hz, 1H), 7.73(d, J=7 Hz, 1 H), 7.51(t, J=8 Hz, 1H), 7.44(d, J=16 Hz, 1H), 7.01(s, 1H), 6.92(dd, J=10 Hz, 2 Hz, 1H), 6.78(d, J=8 Hz, 1H), 6.53(d, J=16 Hz, 1H). FAB-MS (⁺VE) m/z: 370(MH⁺). 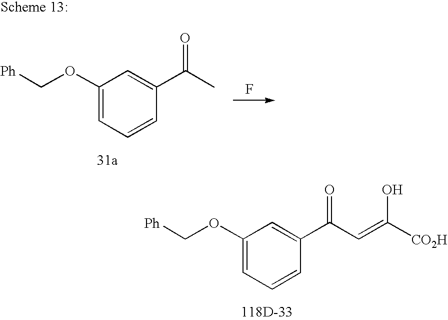
Compound H:

Follow the general procedure F. The beta-diketo acid analogue was obtained as major product.

Yield: 30%. ¹H NMR (DMSO-d₆): δ 7.51-7.65(d, J=8 Hz, 1H), 7.62(d, J=2Hz, 1H), 7.51-7.32(m, 7H), 7.06(s, 1H). FAB-MS (VE) m/z: 297(M−H⁺). 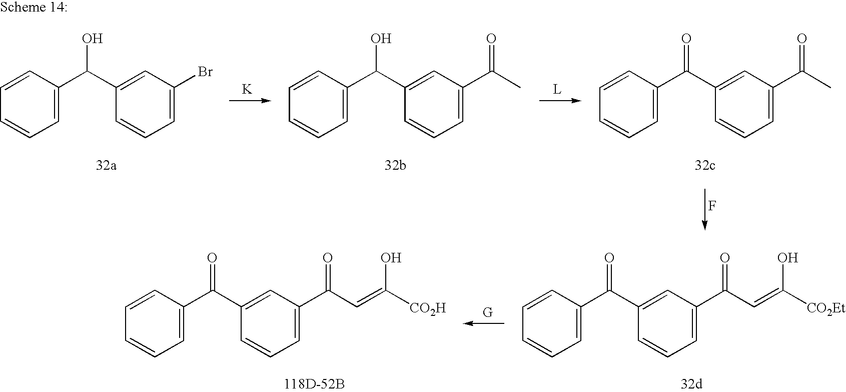
Compound 32b:

Procedure K:

To an oven dried flask was added (3-bromophenyl)phenylmethanol (J Med. Chem. 2000, 4923-4926) (294 mg, 1.12 mmol) in THF 12 ml), the solution was cooled to −78° C. Following dropwise addition of 1.6 M n-butyllithium in hexanes (2.09 ml), the reaction was stirred at −78° C. for 1 h before adding neat N-methoxyl-N-methylacetamide (143 μl, 1.34 mmol). The reaction mixture warmed slowly to room temperature as the bath discharged overnight. The reaction was quenched with 10% KHSO₄ and extracted with ether (3×25ml). The combined organic layers were combined and washed with NaHCO₃, brine and dried over Na₂SO₄. Chrotomographic purification (hexanes: ethyl acetate=4:1-1:1) afforded pure ketone 32b (190 mg, 75%).

Compound 32c. [(a) J. Amer. Chem. Soc. 1998, 4934-4946; (b) J. Amer. Chem. Soc. 1991, 7388-7397]

Procedure L:

Compound 32b (190 mg, 0.84 mmol)was dissolved in acetone (15 ml) and treated with Jone's reagent that was prepared by mixing chromium oxide(26.72 g) with concentrated sulfuric acid (23 ml) followed by water dilution to a final volume of 100 mL. Addition of Jones reagent was continued till the red color persisted for at least 1 min. The resulting mixture was stirred at room temperature for 30 min to ensure the completion of oxidation. The excess oxidizing reagent was quenched by 2-propanol. The solution was then diluted with water followed by repeated extraction with ether. The combined organic extracts were dried over Na₂SO₄, filtered, and concentrated in vacuo. The crude product was purified by flash chromatography (Hexanes:ethyl acetate=5: 1) to give the desire product ( 110 mg, 58%). ,

¹H NMR (DMSO-d₆): δ 7.97(s, 1H), 7.81(dt, J=7.8Hz, 2Hz, 1H), 7.55(d, J=7.5 Hz, 1H), 7.41-7.22(m, 6H), 5.87(s, 1H), 2.55(s, 3H).

Compound 32d. (Tetrahedron Lett. 1992, 4949-4952)

Follow the general procedure F. The mixture containing ketone and alcohol was difficult to be separated and it was used for next step without further purification.

Compound I:

Follow the general procedure G.

Yield: 53%. ¹H NMR (DMSO-d₆): δ8.35(d, J=8.5 Hz, 1 H), 8.29(s, 1H), 8.01(dd, J=6 Hz, 1.5 Hz, 1 H), 7.78-7.57(m, 6H), 7.08(s, 1H). FAB-MS (⁻VE) m/z: 295(M−H⁺).

All patents and publications referred to herein are hereby incorporated by reference for all purposes.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

We claim:

1. The azido diketo carboxylates of formula I: 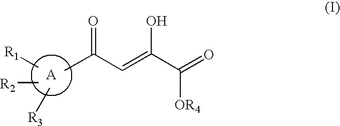
wherein R₁, R₂, and R₃ are independently —H, —N₃, halogen, —OH, —SH, —NH_{2,} —OR₅, —SR₅, —N(R₅)(R₅), a 1 to 6 carbon alkyl, alkene, or alkyne group; and at least one of R₁, R₂, or R₃ comprises an azide moiety;
wherein A is an aromatic ring group;
wherein R₄ is H or 1 to 6 carbon alkyl, alkene, or allyne group;
or a tautomer or pharmaceutically acceptable salt thereof.

2. The azido diketo carboxylate of claim 1, wherein A is an aromatic carbocycle, an aromatic heterocycle, optionally fused to another aromatic ring group or other ring.

3. The azido diketo carboxylate of claim 2, wherein A is a 5 or 6 carbon carbocyclic rings, or a 5 or 6 member heterocyclic ring.

4. The azido diketo carboxylate of claim 3, wherein A is a phenyl ring.

5. The azido diketo carboxylate of claim 3, wherein the 5 or 6 member heterocyclic ring comprises at least one nitrogen atom.

6. The azido diketo carboxylate of claim 5, wherein the heterocyclic ring is a pyrrolyl or pyrazolyl ring.

7. The azido diketo carboxylate of claim 1, wherein R₁, R₂, or R₃ is a 1 to 6 carbon alkyl, alkene, or alkyne group.

8. The azido diketo carboxylate of claim 7, wherein the 1 to 6 carbon alkyl, alkene, or allcyne group comprises —N₃ group.

9. The azido diketo carboxylate of claim 7, wherein the 1 to 6 carbon alkyl, alkene, or alkyne group is substituted.

10. The azido diketo carboxylate of claim 9, wherein the substituents are hydroxyl, sulfhydryl, lower alkyl groups, lower alkoxy groups, lower hydroxyalkyl groups, acyl, allyl, a halogenated alkyl group, —C(O)—, —C(S)—, —C(O)H, ═O, —C(O)OH, or halogen.

11. The azido diketo carboxylate of claim 1, wherein one or more of R₁, R₂, and R₃ is independently —N₃.

12. The azido diketo carboxylate of claim 1, wherein one or more of R₁, R₂, or R₃ is independently a 1 to 6 carbon allcyl substituted with —N₃.

13. The azido diketo carboxylate of claim 1, wherein one or more of R₁, R₂, and R₃ is independently —N₃, —CH₂N₃, or —CH₂CH₂N₃.

14. The azido diketo carboxylate of claim 1, wherein R₄ comprises an N₃ group.

15. The azido diketo carboxylate of claim 1, wherein R₄ is a 1 to 6 carbon alkyl, allcene, or alkyne group.

16. The azido diketo carboxylate of claim 15, wherein the 1 to 6 carbon alkyl, alkene, or alkyne group comprises a —N₃ group.

17. The azido dilceto carboxylate of claim 15, wherein the 1 to 6 carbon alkyl, alkene, or alkyne group is substituted.

18. The azido diketo carboxylate of claim 17, wherein the substituents are hydroxyl, sulfhydryl, lower alkyl groups, lower alcoxy groups, lower hydroxyalcyl groups, acyl, allyl, a halogenated alkyl group, —C(O)—, —C(S)—, —C(O)H, ═O, —C(O)OH, or halogen.

19. The azido diketo carboxylate of claim 1, wherein R₄ is H or a lower alkyl.

20. The azido diketo carboxylate of claim 19, wherein R₄ is methyl or ethyl.

21. A azido diketo carboxylate of claim 1, chosen from 4-(3,5-bis-azidomethyl-phenyl)-2-hydroxy-4-oxo-but-2-enoic, 4-(3-Azidomethyl-phenyl)-2-hydroxy-4-oxo-but-2-enoic acid, or 4-(3-Azido-phenyl)-2-hydroxy-4-oxo-but-2-enoic acid.

22. A method of treating a patient who has, or in preventing a patient from getting, infection by HIV, AIDS, or ARC which comprises administration of a therapeutically effective amount of a azido diketo carboxylate of formula I: 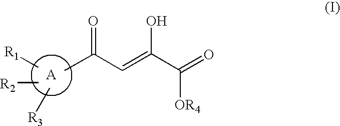
where A, R₁, R₂, R₃, and R₄ are as defined above, or a tautomer or pharmaceutically acceptable salt thereof

23. The method of claim 22, wherein the disease comprises infection by HIV.

24. The method of claim 22, wherein the method of treatment helps to prevent or delay the onset of infection by HIV.

25. The method of claim 22, wherein the disease is AIDS.

26. The method of claim 22, wherein the method helps prevent or delay the onset of AIDS.

27. The method of claim 22, wherein the disease comprises ARC.

28. The method of claim 22, wherein the method helps prevent or delay the onset of ARC.

29. The method of claim 22, wherein the method treats an existing disease.

30. The method of claim 29, wherein the existing disease is HIV, AIDS, or ARC.

31. The method of claim 22, wherein the method prevents a disease from developing.

32. The method of claim 31, wherein the disease that is prevented is HIV, AIDS, or ARC.

33. The method of claim 22, wherein the therapeutically effective amount of the azido diketo carboxylate is administered orally.

34. The method of claim 33, wherein the therapeutically effective amount is from about 0.1 mg/day to about 1,000 mg/day.

35. The method of claim 34, wherein the therapeutically effective amount is from about 1 mg/day to about 100 mg/day.

36. The method of claim 35, wherein the therapeutically effective amount is from about 5 mg/day to about 50 mg/day.

37. The method of claim 22, wherein the therapeutically effective amount of the azido diketo carboxylate is administered parenterally, sublingually, intranasally, or intrathecally.

38. The method of claim 37, wherein the therapeutically effective amount is from about 0.5 to about 100 mg/day.

39. The method of claim 38, wherein the therapeutically effective amount is from about 5 to about 50 mg daily.

40. The method of claim 22, wherein the therapeutically effective amount of the azido diketo carboxylate is administered sublingually, intranasally, or intrathecally.

41. The method of claim 40, wherein the therapeutically effective amount is from about 0.5 to about 100 mg/day.

42. The method of claim 22, wherein the therapeutically effective amount of the azido diketo carboxylate is administered by depo administration, or implants.

43. The method of claim 42, wherein the therapeutically effective amount of the azido diketo carboxylate is from about 0.5 mg/day to about 50 mg/day.

44. The method of claim 22, wherein the therapeutically effective amount of the azido diketo carboxylate is administered topically.

45. The method of claim 44, wherein the therapeutically effective amount of the azido diketo carboxylate is from 0.5 mg/day to about 200 mg/day.

46. The method of claim 22, wherein the therapeutically effective amount of the azido diketo carboxylate is administered rectally.

47. The method of claim 22, wherein the therapeutically effective amount of the azido diketo carboxylate is from about 0.5 mg to about 500 mg.

48. A pharmaceutical composition that comprises a azido diketo carboxylate of formula I: 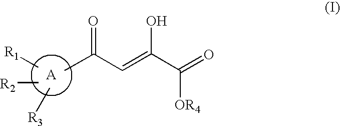
wherein A, R₁, R₂, R₃, and R₄, are as defined above, or a tautomer or pharmaceutically acceptable salt thereof;
and an inert diluent or edible carrier.

49. The use of a azido diketo carboxylate of formula I: 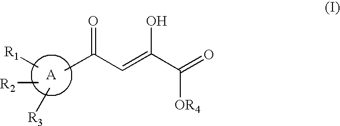
where A, R₁, R₂, R₃, and R₄ are as defined above, or a tautomer or pharmaceutically acceptable salt thereof, for the manufacture of a medicament for use in treating a patient who has, or in preventing a patient from getting, infection by HIV, AIDS, or ARC and who is in need of such treatment.

50. A use according to claim 49, where the disease comprises infection by HIV.

51. A use according to claim 49, where the use helps prevent or delay the onset of infection by HIV.

52. A use according to claim 49, where the disease comprises AIDS.

53. A use according to claim 49, where the use helps prevent or delay the onset of AIDS.

54. A use according to claim 49, where the disease comprises ARC.

55. A use according to claim 49, where the use helps prevent or delay the onset of ARC.

56. A use according to claim 49, employing a pharmaceutically acceptable salt of a azido diketo carboxylate of formula (I).

57. A use according to claim 56, wherein the salt is a base addition salt.

58. A method for inhibiting retroviral integrase activity, that comprises administration of a azido diketo carboxylate of formula (I): 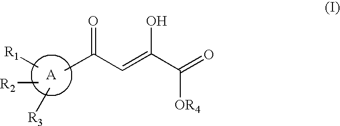
where A, R₁, R₂, R₃, and R₄ are as defined above; or a tautomer or pharmaceutically acceptable salt thereof.

59. A method for inhibiting strand transfer catalyzed by retroviral integrase, that comprises administration of a azido diketo carboxylate of formula (I): 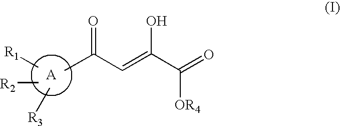
where A, R₁, R₂, R₃, and R₄ are as defined above; or a tautomer or pharmaceutically acceptable salt thereof.

60. A method for inhibiting incorporation of a donor strand DNA into a receiving strand DNA, that comprises administration of a azido diketo carboxylate of formula (I): 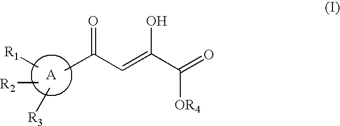
where A, R₁, R₂, R₃, and R₄ are as defined above; or a tautomer or pharmaceutically acceptable salt thereof.

61. A method for inhibiting HIV replication in a cell; for inhibiting HIV replication in an animal, that comprises administration of a azido diketo carboxylate of formula (I): 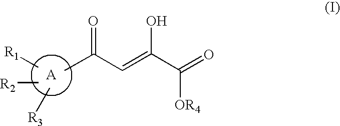
where A, R₁, R₂, R₃, and R₄ are as defined above; or a tautomer or pharmaceutically acceptable salt thereof

62. A method for treating or preventing a disease characterized by HIV infection or replication, that comprises administration of a azido diketo carboxylate of formula (I): 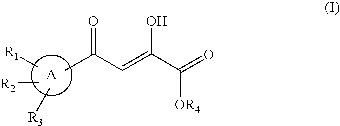
where A, R₁, R₂, R₃, and R₄ are as defined above; or a tautomer or pharmaceutically acceptable salt thereof.

63. A method for inhibiting retroviral integrase activity, comprising exposing the retroviral integrase to an effective inhibitory amount of a azido diketo carboxylate of formula I: 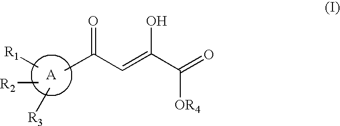
where A, R₁, R₂, R₃, and R₄ are as defined above; or a tautomer or pharmaceutically acceptable salt thereof.

64. A method according to claim 63, wherein the retroviral integrase is exposed t the azido diketo carboxylate in vitro.

65. A method according to claim 63, wherein the retroviral integrase is exposed to the azido diketo carboxylate in a cell.

66. A method according to claim 63, wherein the retroviral integrase is exposed to the azido diketo carboxylate in an animal.

67. A method according to claim 63, wherein the retroviral integrase is exposed to the azido diketo carboxylate in a human.

68. A method for inhibiting strand transfer between a donor DNA strand and a receiving DNA strand, comprising exposing the reaction mixture to an effective inhibitory amount of a azido diketo carboxylate of formula I: 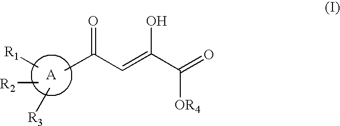
where A, R₁, R₂, R₃, and R₄ are as defined above; or a tautomer or pharmaceutically acceptable salt thereof.

69. The method of claim 68, wherein an integration site recombinant or synthetic DNA is employed as donor and or receiving DNA.

70. The method of claim 68, that employs cellular DNA as receiving DNA.

71. The method of claim 68, wherein the reaction mixture is exposed in vitro.

72. The method of claim 68, wherein the reaction mixture is exposed in a cell.

73. The method of claim 72, wherein the reaction mixture is exposed in an animal cell.

74. The method of claim 73, wherein the reaction mixture is exposed in a human cell.

75. A method for inhibiting HIV replication in a cell, comprising administering to the cell an effective inhibitory amount of a azido diketo carboxylate of formula I: 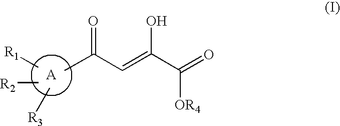
where A, R₁, R₂, R₃, and R₄ are as defined above; or a tautomer or pharmaceutically acceptable salt thereof.

76. The method of claim 75, wherein the effective inhibitory amount is administered to an animal.

77. The method of claim 76, wherein the effective inhibitory amount is administered to a human.

78. A method for inhibiting the replication of HIV or reducing HIV burden in an animal, comprising administering to the animal an effective inhibitory amount of a azido diketo carboxylate of formula I: 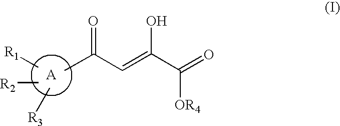
where A, R₁, R₂, R₃, and R₄ are as defined above; or a tautomer or pharmaceutically acceptable salt thereof.

79. The method of claim 78, wherein the effective inhibitory amount is administered to a human.

80. A method for treating or preventing a disease characterized by HIV integration or replication comprising administering to a patient an effective therapeutic amount of a azido diketo carboxylate of formula I: 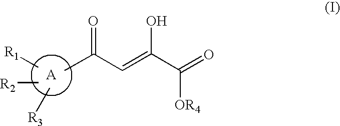
where A, R₁, R₂, R₃, and R₄ are as defmed above; or a tautomer or pharmaceutically acceptable salt thereof.

81. The method of claim 80, wherein the effective therapeutic amount of a azido diketo carboxylate of formula (I) is in the range of from about 0.1 to about 1000 mg/day.

82. The method of claim 81, wherein the effective therapeutic amount of a azido diketo carboxylate of formula (I) is in the range of from about 15 to about 1500 mg/day.

83. The method of claim 81, wherein the effective therapeutic amount of a azido diketo carboxylate of formula (I) is in the range of from about 1 to about 100 mg/day.

84. The method of claim 83, wherein the effective therapeutic amount of a azido diketo carboxylate of formula (I) is in the range of from about 5 to about 50 mg/day.

85. The method of claim 80, wherein the disease comprises AIDS.

86. The method of claim 80, wherein the disease comprises HIV infection.

87. A composition comprising retroviral integrase complexed with a azido diketo carboxylate of formula I: 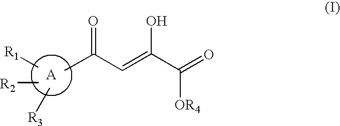
where A, R₁, R₂, R₃, and R₄ are as defined above; or a tautomer or pharmaceutically acceptable salt thereof.

88. A method for producing a retroviral integrase complex comprising exposing retroviral integrase to a azido diketo carboxylate of formula I: 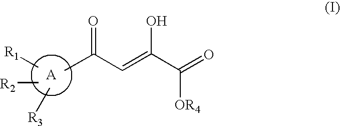
where A, R₁, R₂, R₃, and R₄ are as defined above; or a tautomer or pharmaceutically acceptable salt thereof, in a reaction mixture under conditions suitable for the production of the complex.

89. The method of claim 88, wherein the retroviral integrase is exposed to the azido diketo carboxylate of formula (I) in vitro.

90. The method of claim 88, wherein the reaction mixture is a cell.

91. A component kit comprising component parts capable of being assembled, in which at least one component part comprises a azido diketo carboxylate of formula (I) according to claim 1, enclosed in a container.

92. The component kit of claim 91, wherein at least one further component part comprises a diluent.

93. The component kit of claim 92, further comprising a lyophilized azido diketo carboxylate.

94. A container kit comprising a plurality of containers, each container comprising one or more unit dose of a azido diketo carboxylate of formula I: 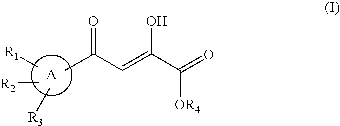
where A, R₁, R₂, R₃, and R₄ are as defined above; or a tautomer or pharmaceutically acceptable salt thereof.

95. The container kit of claim 94, wherein each container is adapted for oral delivery.

96. The container kit of claim 95, wherein each container contains a tablet, gel, or capsule.

97. The container kit of claim 94, wherein each container is adapted for parenteral delivery.

98. The container kit of claim 97, wherein each container contains a depot product, syringe, ampoule, or vial.

99. The container kit of claim 94, wherein each container is adapted for topical delivery.

100. The container kit of claim 99, wherein each container contains a patch, medipad, ointment, or cream.

101. An agent kit comprising a azido diketo carboxylate of formula I: 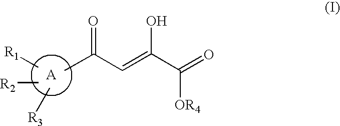
where A, R₁, R₂, R₃, and R₄ are as defined above; or a tautomer or pharmaceutically acceptable salt thereof, and one or more therapeutic agents.

102. The agent kit of claim 101, wherein the therapeutic agent is nucleoside analog reverse transcriptase inhibitors, non-nucleoside reverse transcriptase inhibitors, protease inhibitors, other antivirals, immunomodulators, or anti-infectives.

103. A composition comprising: a azido diketo carboxylate of formula I: 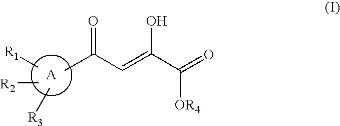
where A, R₁, R₂, R₃, and R₄ are as defined above; or a tautomer or pharmaceutically acceptable salt thereof; and an inert diluent or edible carrier.

104. The composition of claim 103, wherein said carrier comprises an oil.

105. A composition comprising: a azido diketo carboxylate of formula I: 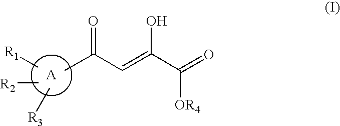
where A, R₁, R₂, R₃, and R₄ are as defined above; or a tautomer or pharmaceutically acceptable salt thereof; and a binder, excipient, disintegrating agent, lubricant, or gildant.

106. A composition comprising: a azido diketo carboxylate of formula I: 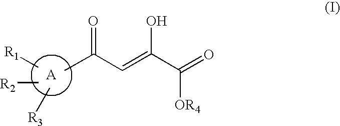
where A, R₁, R₂, R₃, and R₄ are as defined above; or a tautomer or pharmaceutically acceptable salt thereof; disposed in a cream, ointment, or patch.
